(19) United States
(12) Patent Application Publication
(10) Pub. No.: US 2009/0117634 A1
(43) Pub. Date: May 7, 2009

(54) Process of Producing Ethanol Using Cellulose with Enzymes Generated Through Solid State Culture

(75) Inventors: Clifford BRADLEY, Missoula, MT (US); Robert KEARNS, Melrose, MT (US)

Correspondence Address: MORGAN, LEWIS & BOCKIUS, LLP, ONE MARKET SPEAR STREET TOWER, SAN FRANCISCO, CA, 94105, US

(73) Assignee: ENERGY ENZYMES, INC., Cupertino, CA (US)

(21) Appl. No.: 12/264,857

(22) Filed: Nov. 4, 2008

Related U.S. Application Data
(60) Provisional application No. 60/985,408, filed on Nov. 5, 2007.
(60) Provisional application No. 60/985,430, filed on Nov. 5, 2007.
(60) Provisional application No. 60/985,452, filed on Nov. 5, 2007.
(60) Provisional application No. 61/021,211, filed on Jan. 15, 2008.
(60) Provisional application No. 61/024,339, filed on Jan. 29, 2008.
(60) Provisional application No. 61/097,169, filed on Sep. 15, 2008.

(51) Int. Cl. C12P 7/10; C12N 9/42

(52) U.S. Cl. 435/165; 435/209

(57) ABSTRACT

The present invention is directed to process of producing ethanol using cellulose with enzymes generated through solid state culture.

### CROSS REFERENCE TO RELATED APPLICATIONS

This application claims the benefit of, and priority to, U.S. Provisional Patent Application Ser. Nos. 60/985,452, 60/985,430, and 60/985,408 filed on Nov. 5, 2007, 61/021,211, filed on Jan. 15, 2008, 61/024,339 filed on Jan. 29, 2008, and 61/097,169 filed on Sep. 15, 2008, the entire disclosures of which are hereby incorporated by reference in their entireties.

### FIELD OF THE INVENTION

The present invention is directed to compositions of cellulase enzymes generated through solid substrate culture and the process of using these enzymes in producing ethanol from cellulose containing feedstocks.

### BACKGROUND OF THE INVENTION

One of the renewable alternative energy sources are biofuels converted from biomass. Of many of substitutes to gasoline, one of the most generally recognized substitutes which could be made available in significant quantities in the near future is alcohol, and in particular, ethanol. For example, there are currently many outlets in the United States and throughout the world which sell a blend of gasoline and about 10%- to 20% ethanol (commonly called “gasohol”) which can be used as a fuel in conventional automobile engines. Furthermore, ethanol can be blended with additives to produce a liquid ethanol-based fuel, with ethanol as the major component, which is suitable for operation in most types of engines. Ethanol can be produced from almost any material which either exists in the form of, or can be converted into, a fermentable sugar. There are many natural sugars available for fermentation, but carbohydrates such as starch and cellulose can be converted into fermentable sugars which then are fermented into ethanol.

Currently, most ethanol is produced from starch in corn grain using amylase enzymes to degrade the starch to fermentable sugars. In general, while the corn grain is used in the production of ethanol, the remainder of the corn biomass, i.e., the leaves and stalks, is seldom used because of the cost in degrading the leaves and stalks comprising lignins, hemicellulose and cellulose, generally in the form of lignocellulose, to fermentable sugars. The lignocellulose in the stalks and leaves of corn as well as many other forms of lignocellulsic biomass represents a tremendous source of untapped energy that goes unused because of the difficulty and cost of converting it to fermentable sugars. There are also abundant sources of cellulose that could be tapped into to produce ethanol, such as paper mill waste and grass.

Currently, there are four technologies available to convert cellulose to fermentable sugars. These are concentrated acid hydrolysis, dilute acid hydrolysis, biomass gasification and fermentation, and enzymatic hydrolysis.

Concentrated acid hydrolysis is based on concentrated acid de-crystallization of cellulose followed by dilute acid hydrolysis to sugars at near theoretical yields. Separation of acid from sugars, acid recovery, and acid re-concentration are critical unit operations.

Dilute acid hydrolysis occurs in two stages to maximize sugar yields from the hemicellulose and cellulose fractions of biomass. The first stage is operated under milder conditions to hydrolyze hemicellulose, while the second stage is optimized to hydrolyze the more resistant cellulose fraction. Liquid hydrolyzates are recovered from each stage, neutralized, and fermented to ethanol.

In biomass gasification and fermentation, biomass is converted to a synthetic gas, which consists primarily of carbon monoxide, carbon dioxide, and hydrogen via a high temperature gasification process. Anaerobic bacteria are then used to convert the synthetic gas into ethanol.

In early processes embracing enzymatic hydrolysis of biomass to ethanol, the acid hydrolysis step was replaced with an enzyme hydrolysis step. This process scheme was often referred to as separate hydrolysis and fermentation (SHF). Wilke et al., Biotechnol. Bioengin. 6: 155-175 (1976). In SHF, pretreatment of the biomass is required to make the cellulose more accessible to the enzymes. Many pretreatment options have been considered, including both thermal and chemical steps.

An important process improvement made for the enzymatic hydrolysis of biomass was the introduction of simultaneous saccharification and fermentation (SSF). See U.S. Pat. Nos. 3,990,944 and 3,990,945. This process reduced the number of reactors involved by eliminating the separate hydrolysis reactor and addressing the problem of product inhibition associated with enzymes.

In the presence of glucose, β-glucosidase stops hydrolyzing cellobiose. The build up of cellobiose, in turn, shuts down cellulose degradation. In the SSF process scheme, cellulase enzymes and fermenting microbes are combined. As sugars are produced by the enzymes, the fermentative organisms convert them to ethanol. The SSF process has been improved to include the co-fermentation of multiple sugar substrates in a process known as simultaneous saccharification and co-fermentation (SSCF).

Cellulase enzymes are already commercially available for a variety of applications. However, most of these applications do not involve extensive hydrolysis of cellulose. For example, the textile industry applications for cellulases require less than 1% hydrolysis. Ethanol production, by contrast, requires nearly complete hydrolysis. In addition, most of the commercial applications for cellulase enzymes represent higher value markets than the fuel market. For these reasons, enzymatic hydrolysis of biomass to ethanol remains non-competitive.

Commercial cellulase enzymes generally do not contain all of the activities necessary to completely convert both the cellulose and hemicellulose. Different enzyme preparations produced in separate fermentations of different organisms are necessary to efficiently hydrolyze both the cellulose and hemicellulose to monomeric C6 and C5 sugars. Thus, there is still a need for compositions and methods to simplify the ethanol production process from cellulose and to reduce the cost. In particular there is a need to produce low cost preparations that contain multiple hemicellulase and cellulase enzyme activities.

### BRIEF DESCRIPTION OF THE DRAWINGS

FIG. 1 depicts the scheme of using solid state culture to produce cellulose/hemicellulose enzyme composition.

FIG. 2 depicts the ethanol production process using cellulose material without lignin.

FIG. 3 depicts the ethanol production process using straw. Screw press is a device to separate solids from liquids in which a screw or auger forces a suspension of solids in liquid through a screen, retaining the solids and pressing the liquid fraction through the screen.

FIG. 4 depicts a system for ethanol production from cellulose.

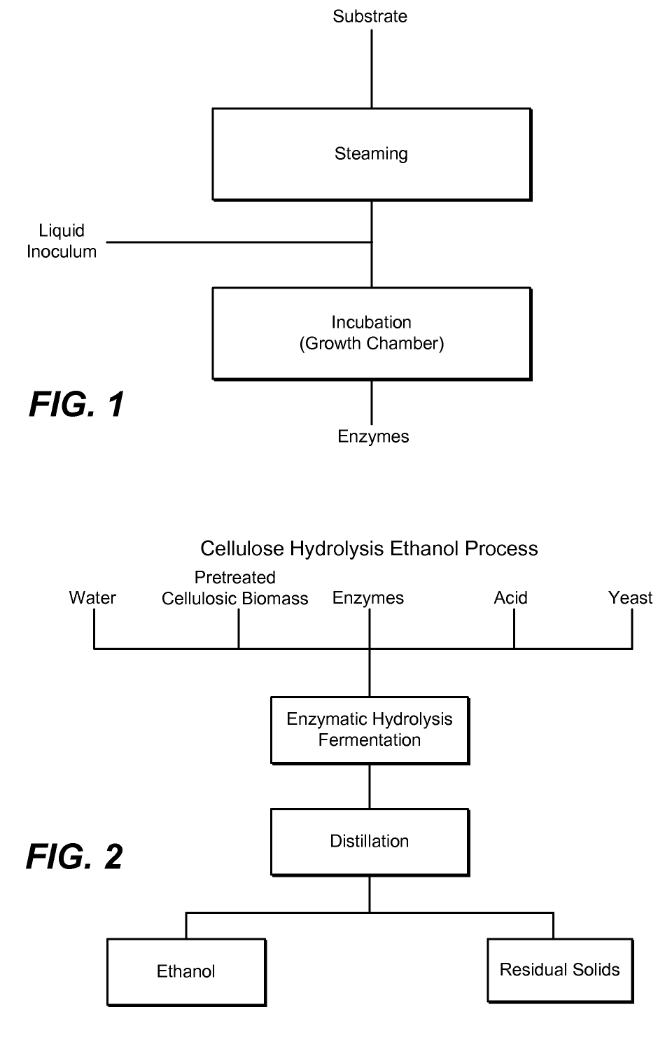

### DETAILED DESCRIPTION OF THE INVENTION

In general, the present invention relates to methods and compositions to produce enzymes for use in producing ethanol from cellulose. To that end, the present invention provides methods to produce low cost enzyme preparations that contain a mixture of cellulase and hemicellulose enzymes that are used to convert cellulose into sugar, which is further fermented to produce ethanol. The present invention uses solid substrate culture to produce an enzyme preparation from one organism in one culture incubation that contains all of the activities necessary to produce monomeric C5 and C6 sugars from pretreated lignocellulosic feedstocks. Solid substrate culture (SSC) enzyme preparations of the invention contain endo glucanase, cellobiohydrolase, celliobiase and xylanase activities. The enzyme preparations of the present invention may be used to produce fermentable sugars from any source of lignocellulose that has been optionally pretreated using any of the common pretreatment methods to dissociate lignin. Such pretreatment methods include but are not limited to hot water, dilute acid, dilute alkali, alkaline peroxide, steam explosion, and/or ammonia explosion (AFEX), alone or in any combination.

One advantage of the process provided herein is to produce low cost, high concentration, mixed activity cellulose/hemicellulase enzyme preparations. The enzyme preparation provided herein can be used to hydrolyze cellulose and hemicellulose in any delignified lignocellulose to fermentable sugars. The enzyme preparation can be used with any pretreatment process for lignification including dilute alkaline process which generally do not hydrolyze hemicellulose. The SSC cellulases provided herein can also be used in any fermentation process including sequential hydrolysis and fermentation, or simultaneous hydrolysis and fermentation.

The SSC cellulase enzyme preparation of the present invention is of practical importance to ethanol production from lignocellulose feedstocks. The enzyme preparation generally includes a mixture of cellulase and hemicellulase activities. However, the preparation generally also includes high concentration of enzyme activities in the residual culture substrate. This generally eliminates the need to concentrate or purify the enzyme activities to obtain economically and technically practical enzyme doses.

The process of producing enzymes by growing a fungus on solid culture begins with selection of the proper fungus and substrate.

In one aspect, the present invention provides a strain of Trichoderma reesei (also known as Hypocrea jecorina) that can be used in methods of the invention. In another aspect, the present invention provides growth substrates and growing conditions that allow production of enzyme preparations using fungus, such as the strain of Trichoderma provided herein. The invention can be generally described as follows. The substrate is selected to provide nutrition for fungal growth and the physical structure of the solid substrate culture. The dry substrate is moistened with added water or a nutrient containing solution then steamed to adjust moisture and reduce contamination from indigenous microorganisms. The steamed substrate is inoculated with the desired fungus and loaded into a solid support growth chamber. The final moisture content of the substrate is such that the moisture is absorbed into the substrate and the substrate remains solid. The fungus grows on the substrate, utilizing it as a nutrient source, and at the same time producing the desired enzymes. The incubation time varies depending on the enzymes being produced. After the incubation, the whole culture is harvested to obtain the enzyme preparation. In some embodiments, the whole culture is used for converting cellulose to sugar and no additional purification of the enzymes is required. Alternatively the enzymes can be extracted and purified from the culture substrate. These enzyme preparations called, referred herein as SSC Cellulase, can be used in any process for enzymatic hydrolysis and/or enzymatic hydrolysis and fermentation of lignocellulose. Generally, the preferred process is a simultaneous hydrolysis of cellulose and fermentation conducted at the upper temperature limit of the yeast, generally about 35° C. and the pH optima of the enzyme about pH 4.8. In another aspect, the present invention provides methods for fermenting sugar into ethanol.

The process of converting delignified lignocellulose to ethanol requires hydrolysis of both the cellulose and hemicellulose fractions and can be summarized as the following:

The first step is the hydrolysis of cellulose:

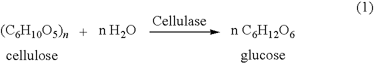

The second step is the conversion of glucose to ethanol:

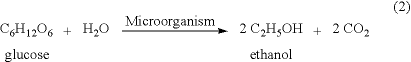

The first step of cellulose hydrolysis can be further broken into two steps:

One step is the conversion of cellulose to cellobiose:

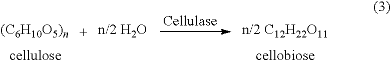

This is followed by a step of converting cellobiose to glucose:

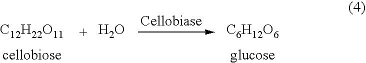

The process of hemicellulase (such as xylanse) hydrolysis followed by fermentation is the follows:

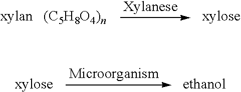

Thus, the present invention provides a process comprising enzyme production using solid substrate culture, the composition of an enzyme preparation containing multiple cellulase and hemicellulase activities and a second step to produce ethanol.

### I. Strain Selection

In one aspect of the present invention, a strain of Trichoderma reesei obtained from a public collection (USDA) is used for growth on cellulose at low pH. When grown in the SSC process as provided herein this strain produces a mixture of enzymes that hydrolyze the cellulose at an optimum pH of 4.8.

Trichoderma reesei is a mesophilic and filamentous fungus, the anamorph of Hypocrea jecorina. It has the capacity to secrete large amounts of cellulolytic enzymes (cellulases and hemicellulases). A detailed description of the Trichoderma reesei strain used in the present invention is provided in Example 1. The strain is ATCC-56765.

By “strain” herein is meant a genetic variant or subtype of a fungus. Thus, there is genotype and/or phenotype difference between a strain and the parent strain from which it is derived. The creation of a new strain can due to either naturally occurred mutations or artificially introduced mutations.

Other fungus that can produce cellulases and or hemicellulases may also be used in the present invention. Suitable fungal species include other strains of T. reesei, Aspergillus niger, A. phoenicis, A. oryzae, A awamori, Rhizopus oryzae, R. microsporus, Acidothermus cellulyticus and Trichoderma koningii, Trichoderma viride. T. harzianum, Fusarium oxysporum Penicillum pupurogenum Myceliophthora sp., Lentinous. Other suitable organisms are listed in Pandey et al., Current Science (Bangalore), 77(1):149-163 (1999), herein incorporated by reference.

### II. Solid Substrate Culture Technology

The instant application further provides solid substrate culture technology (sometimes referred to as solid state fermentation or solid state culture) to produce enzyme preparations capable of converting cellulose to glucose and hemicellulose to monomercic C5 and C6 sugars, including xylose, arabinose and galactose.

Conventional ethanol processes uses enzymes produced in liquid fermentation. In general, these are specific enzymes that are purified and concentrated. Furthermore, when more than one enzyme is used in an ethanol process (e.g., hydrolysis of cellulose to glucose), the individual enzymes are generally produced in separate liquid fermentation vessels. One of the primary costs of enzymes produced in liquid culture is the cost of concentrating the enzymes or separating the enzymes from the broth in which they are generated. The more liquid in the process, the higher transportation, storage and distillation costs. The present invention provides new methods of producing multiple enzymes in the same culture at high concentrations, and generally, does not require post-production purification.

Accordingly, the instant invention provides a solid substrate culture technology that results in enzyme preparations produced from one organism with high concentrations multiple enzyme activities that work effectively in downstream ethanol production from cellulose.

By “solid substrate culture (SSC)” or “solid state fermentation (SSF)” herein is meant a culture wherein the organism is grown on the surface of a moist solid material where a majority (or in some cases, all) of the water is absorbed into the substrate material. Thus, there is a minimum amount of water or substantially no free water in the culture, which facilitates handling, and minimizes bacterial contamination among other things. The substrate material provides both the nutrients and physical support for the culture. The organism obtains oxygen from the air or from modified atmosphere introduced into the growth chamber. Depending on the composition and water sorbancy of the substrate, the substrate moisture can range from 30 to 90% (w/w) final moisture content. In the present invention the substrate moisture generally ranges from about 50 to 80% with an optimum about 65 to 70%.

Solid substrate culture is different from conventional liquid fermentation. In a liquid fermentation system, a microorganism is placed in a liquid environment that contains soluble nutrients. Air or oxygen is bubbled through the liquid using agitation or injection to dissolve oxygen in the liquid. Generally there is not any solid support media and the oxygen available to the organism is limited by the solubility of oxygen in water.

Solid culture technology has been around for over a hundred years. Most applications of solid culture technology involve the use of specific substrates or nutrients to achieve a specific end product. Sake and soy sauce are good examples. For review, see Pandey et al., Current Science (Bangalore), 77(1):149-163 (1999), herein incorporated by reference. These applications usually involve a high degree of sterilization since humans consume the resulting product. They typically use very thin layers of substrate on trays and involve a lot of material handling to prepare the substrate, grow the appropriate fungus and recover the end product. Generally, food applications justify the cost of production associated with these processes.

Mushrooms are another example of solid culture technology. Mushrooms are slow growing (compared to the fungal strains used herein) and therefore do not generate much metabolic heat. Control of temperature in mushroom compost is fairly simple as is harvest of the mushrooms.

One of advantage of the SSC systems provided herein is that it mimics nature. In nature, fungi grow on moist damp surfaces, with atmospheric oxygen concentration, not in liquids. In the SSC system provided herein, when a selected fungus is grown on the proper solid nutrient source, it often produces a set of enzymes that are functionally different than the enzymes it would produce when grown in a liquid culture. If these fungal strains were produced using conventional liquid fermentation technology, they would generally not produce the same enzyme complex and could be ineffective to convert cellulose to sugar at ambient temperatures.

In one aspect, the present invention provides process for fungal culture and enzyme preparation employing solid substrate culture. The present invention enables sufficient large scale solid substrate culture.

The present invention provides innovations in physical and biochemical substrate characteristics and process control that reduce costs and improve efficiency of large scale solid state culture. Substrate characteristics induce high product concentrations using low cost materials in large volume cultures, (e.g., up to ten tons of dry weight substrate in a single culture reactor). The present invention also provides methods to control temperature and moisture balance in large scale cultures with very rapid generation of metabolic heat. The selected fungal strain produces multiple cellulase and hemicellulase enzymes when grown in these solid state cultures.

In another aspect, the present invention provides enzyme preparations used in conversion of cellulose to ethanol. The selected fungal strain provide herein is grown in solid state culture to produce an enzyme preparation containing multiple enzyme activities that act on a variety of cellulose and hemicellulose substrates, producing fermentable sugars (for example, glucose and xylose). The enzyme preparation can be used in multiple-step process, where the enzyme preparation is first used to convert cellulose and hemicellulose to sugars, and in a second step the sugars are fermented into ethanol; this is referred to as a separate or “two-step process.” In the separate process the glucose and the five carbon sugars from the hemicellulose hydrolysis may be co-fermented using one yeast strain or fermented separately using different fermentative organisms. Alternatively, the fermentation process step can start before all cellulose is converted into sugar, thus there is some overlap between the cellulose hydrolysis step and the fermentation step. In some embodiments, as described in more detail herein, the enzyme preparation is used in a simultaneous cellulose hydrolysis and fermentation process which combines cellulose, hemicellulose, enzyme, and yeast in a single tank to produce ethanol (referred to herein as a “one-step process.”)

As described below, in some embodiments, the enzyme preparation provided herein comprises the whole solid substrate fungal culture including residual substrate, fungal cells and protein enzymes. When the culture reaches optimal enzyme concentration, the whole culture is harvested. The culture may be used wet without any further processing or may be dried and stored for later use. The culture is a whole culture enzyme preparation containing multiple enzyme activities. The combination of the selected fungal strain and solid substrate culture technology produces sufficiently high enzyme titers that no further processing is required to reach usable enzyme concentrations. This eliminates the principal cost in producing enzymes in conventional liquid fermentation.

Furthermore, exogenous cellulase (for example from different fungus) can also be added to the enzyme preparation.

In another aspect of the present invention, the enzyme preparation provided herein can be further purified, or partially purified, to produce enzymes with higher purity or activities. It can also be used to purify specific enzymes with enzyme purification technologies known in the art.

A. Substrate Selection and Preparation

The process of producing enzymes by growing a fungus on solid culture begins with the selection of the proper fungus and substrate. The selected fungi should be able to metabolize cellulose.

There are known methods of growing fungus on solid substrate; see for example, Ellaiah P. et al. Process Biochemistry, 38(4):615-620 (2002); U.S. Pat. No. 6,558,943.

The present invention provides solid state culture substrates with moisture retention capability and physical strength to use in a packed bed without collapsing or “mushing down”. These solid substrate culture substrates are processed to provide a material with both the physical and nutritional requirements necessary for optimal fungal growth and enzyme production. Some additional soluble nutrients are added to achieve the desired fungal growth and enzyme complex.

Many different solid substrates can be used for the production of enzymes using fungus, such as the production of cellulase employing Trichoderma reesei under solid state fermentation. These include, but are not limited to, wheat straw, wheat bran, corn stalks, switchgrass, wood chips, saw dust, green gram straw, black gram straw, barley straw, oat straw, rice straw, rice husks, sugar cane bagasse, sugar beet pulp, apple pomice, and coffee process waste. In some embodiments, the material used is called BPC (for beeswing, pith and caffe), a fraction of corn cobs from Mount Pulaski Products, Mount Pulaski IL. This material provides cellulose and hemicellulose as a carbon source, structure to the substrate and water holding capacity. BPC is very water sorbant. Other corn cob fractions such as cob meal which is finely ground whole cob also work. Substrates are generally moistened with water and steam sterilized.

In general, the substrate comprises a mixture of components. The mixture of materials used in the composition of SSC substrate was developed to provide: (1) suitable physical characteristics; (2) nutrition of the fungal growth, and (3) production of the desired mixture of enzyme activities. Substrate ingredients were also selected because they are low cost and readily available in large amounts.

In one aspect, the substrate provided in the present invention comprises a component that provides structural strength and/or moisture reservoir or buffer. Many other cellulose containing materials could also be used, including but not limited to, corn cob fractions and straw. Corn cob fractions and straw provide physical structure as well as a lost cost means of controlling water activity. In some embodiments corn cob fractions are preferred because of very high water sorbancy and low cost. The corn cob fractions generally absorb three times their weight in water and remain a friable solid. In addition, it was observed that the selected fungal strain grew on cob meal, BPC or straw, but the growth rates were slower and produced lower enzyme activity.

In many embodiments, the substrate comprises fractions of corncobs. Corncob is the central wooden core of a maize ear. The majority of a corncob is composed of cellulose (lignocellulose and hemicellulose). Corncob meal, which is obtained by drying and crushing corncobs, is used as a fungal bed for growing mushrooms. Corncobs not only provide a source of cellulase, but also provide both structural strength and a moisture reservoir or buffer. Suitable corncob substrates can be obtained from a variety of sources.

Approximately 60% of the corncob's weight is made up of hard woody ring. This portion is not a good absorber of water soluble substances. The pith and chaff portion of the corncob are the lighter components that make up the balance of the corncob weight. In their loose form, after having been reduced, for example, by grinding rolls and a hammer mill, these lighter ends can absorb in excess of 350% of their weight in some oils and water and water based liquids. Such a loose, lighter corncob product of chaff and pith which has been separated from the hard woody ring is produced by The Andersons in Maumee, Ohio and is marketed under the trademark SLIKWIK™ (Now owned by Sorbent Products Co. Inc.).

In some embodiments, sugar cane bagasse or fractions of bagasse is used to reduce or replace corn cob fractions.

The percentage of corncobs in the total substrate can be from 30 to 80% (w/w), preferably from 45 to 70%, and even more preferably from 45 to 50% (w/w).

In another aspect, the substrates provided herein for enzyme production comprise a component that provides cellulose as carbon source, such as straw, corn stover, wood chips, and switchgrass.

Switchgrass (Panicum virgatum) is a warm season grass and is one of the dominant species of the central North American tallgrass prairie. Switch grass can be found in remnant prairies, along roadsides, pastures and as an ornamental plant in gardens. Other common names for this grass include tall panic grass, Wobsqua grass, lowland switch grass, blackbent, tall prairie grass, wild redtop and thatchgrass.

By “straw” herein is meant the dry stalk of a cereal plant after the nutrient grain or seed has been removed. Straw makes up about half of the yield of a cereal crop such as barley, oats, rice, rye or wheat.

In some embodiments grass or grain straw milled to a particle size ranging from 5 mm down to a fine flour is incorporated into the substrate. The percentage of straw may range from 1 to 15% preferably, 3 to 10% and more preferably 5%.

In one aspect, the substrate comprises a component that provides an inducer of cellulase production in the fungus. The biosynthesis of cellulases is induced by cellulose, cellobiose, sophorose and lactose; and repressed by glucose or other readily utilizable carbon sources. The type of inducer that can be used in the present invention depends on the type of fungus that used in the present invention for the production of cellulose. The inducers can be any one known in the art, including, but not limited to, cellulose, lactose, cellobiose, sorbose, cellobionolactone, lactobionic acid, lactulose, and β-glucan, including monosaccharides and disaccharides. It can also be one that is uncovered in an assay for inducer of cellulose production by a given fungus.

A variety of methods can be used for screening for an inducer or test an inducer. For example, to screen for an inducer, a candidate inducer is added to a culture of a cellulase generating microorganism, such as Trichoderma reesei. After continuous cultivation for a suitable period of time (for example, for 96-120 h), cells are separated (for example, by centrifugation at 4° C.) and the obtained supernatant is used for enzyme analyses. The activity of cellulases (FPU) of culture filtrates can be assayed according to the method such as that described by Mandels et al., Measurement of Saccharifying Cellulase. Biochim. Bioeng. Symp., 6:21-23 (1976), and expressed in International Unit (IU), using Whatman No. 1 filter paper. The amount of inducer to be added to the culture can be varied, and preferably a broad range of amount can be used for the screening, such as to achieve a final concentration of 0.1% to 1% (v/v) in the culture. See also, Janas et al., New Inducers For Cellulases Production by Trichoderma Reesei M-7, Electronic Journal of Polish Agricultural Universities, Food Science and Technology, 5(1) (2002) (available online, web address: www.ejpau.media.pl/series/volume5/issue1/food/art-04.html), herein incorporated by reference.

In some embodiments, the inducer is β-glucan. A glucan molecule is a polysaccharide of D-glucose monomers linked by glycosidic bonds. Some of the commonly known glucans include: cellulose (β-1,4-glucan), laminarin (β-1,3- and β-1,6-glucan), starch (α-1,4- and α-1,6-glucan), glycogen (α-1,4- and α-1,6-glucan), and dextran, (α-1,6-glucan). β-glucans (or beta-glucans) are natural gum polysaccharides occurring in the bran of cereal grains, most abundantly in barley and oats and to a much lesser degree in rye and wheat. In many embodiments of the invention short chain soluble beta 1,3 linked glucans act as inducers of cellulase activities.

The inducer added to the substrate can be in a relative pure form. For example, it can be synthesized chemically, or purified (completely or partial) from a source material. The inducer can also be from a natural source without purification. For example, when β-1,3 glucan is used as an inducer of cellulose production, barley can be used as the source without any purification. Alternatively, an inducer can be provided by genetic engineered microorganisms, such as bacteria, but preferably fungi, that can produce such an inducer. The inducers can be added individually, or in combination to achieve better effects. For example, although lactose alone induces little cellulase under certain conditions, a synergistic effect on cellulase formation was observed following the addition of sophorose, cellobiose or galactose to lactose. Morikawa Y. et al., Cellulase induction by lactose in Trichoderma reesei PC-3-7, Applied Microbiology and Biotechnology, 44(1-2):106-111 (1995), herein incorporated by reference.

Thus, in some embodiments, any material that can provide soluble beta 1,3 linked glucans can be used in the present invention. One of such material is barley. Barley (Hordeum vulgare) is a cereal grain, which serves as a major animal feed crop, with smaller amounts used for malting and in health food. It is a member of the grass family Poaceae. In 2005, barley ranked fourth in quantity produced and in area of cultivation of cereal crops in the world (560,000 km²). Barley not only acts as source of nutrition (such as in the form of cellulose and starch), but also may act as an inducer for cellulase and hemicellulase. There is high concentration of beta glucans in barley. Barley may be used in different forms: whole barley ground through a mill to a course or fine powder, barley flour which is barley grin that is dehulled then ground to a fine flour, barley pearling waste which is the hull and a portion of the kernel removed during preparation of pearled barley. In some embodiments, barley and/or wheat germ is added to accelerate fungal growth and induce production of high concentrations of cellulase/hemicellulase activities. In some embodiments, additional inducer, including but not limited to lactose, is added in addition to barley.

The percentage of barley in the total substrate can be from 10 to 60% (w/w), preferably from 25 to 50%, and even more preferably is about 40%.

In another aspect, substrate provided herein comprises at least one nutrient supplement such as wheat germ. Wheat germ is the vitamin-rich embryo of the wheat kernel. It is generally separated before milling for use as a cereal or food supplement. Wheat germ not only provides as carbon source, but also provides nutrients, such as vitamin and amino acids, which contribute to robust fungal growth.

The percentage of wheat germ in the total substrate can be from 1 to 20% (w/w), preferably from 5 to 15%, and even more preferably is about 10%.

Other additives can also be included, such as soluble nutrients, and minerals can be dissolved in the water used to wet the substrate. Such solutions promote fungal growth but water alone is sufficient for cellulase/hemicellulase production by the fungus. Different kind of salts can be used, such as those generally used as component in culture medium for growing microorganisms, including, but not limited to, ammonium sulfate, potassium phosphate, magnesium chloride, calcium chloride, and trace minerals including ferric chloride, manganese chloride, cobalt chloride, zinc chloride, copper chloride and soluble protein/nitrogen source such as urea, peptone or yeast extract. The nutrient solution added to substrate in the preferred embodiment contains the following in grams per liter: ammonium sulfate 11.6, potassium phosphate 3.8, magnesium chloride 0.3, calcium chloride 0.6, urea 0.6, soy peptone 2.9 and trace minerals each with less than or equal to 0.1 g/liter including ferric chloride, manganese chloride, cobalt chloride, zinc chloride, copper chloride.

Then the components are mixed together, such as by mechanical methods.

In many embodiments, the pH of the substrate is also adjusted to low pH as described herein. The pH can be from 3 to 7, preferably from 4 to 6, and more preferably is about 4.5 to 5.

Many different chemicals can be used to adjust the pH, such as ammonia and acid, the later including, but not limited to, sulfuric acid, phosphoric acid acetic acid, lactic acid, citric acid, and hydrochloric acid. The mixing of the substrate and the adjusting of the pH can be carried out in a single step, or in separate steps.

As used herein, the term “about” modifying any amount refers to the variation in that amount encountered in real world conditions of producing sugars and ethanol, e.g., in the lab, pilot plant, or production facility. For example, an amount of an ingredient employed in a mixture when modified by “about” includes the variation and degree of care typically employed in measuring in an ethanol production plant or lab. For example, the amount of a component of a product when modified by “about” includes the variation between batchs in an ethanol production plant or lab and the variation inherent in the analytical method. Whether or not modified by “about,” the amounts include equivalents to those amounts. Any quantity stated herein and modified by “about” can also be employed in the present invention as the amount not modified by “about.”

The substrate components used herein can be processed or raw agriculture products. Many agricultural products, particularly raw products, have indigenous microbial contamination. Left untreated, these contaminants can compete, and potentially out-compete the desired fungi, resulting in a contaminated product, low quality product, or no useable product. As is known in the art, there may be a variety of techniques used to reduce the contamination, including, but is not limited to, heat treatment, steaming, radiation, and treatment with antibiotics.

Steaming finds particular use in the present invention. By “steaming” herein is meant the process of applying vaporized liquid (usually water, although other aqueous solutions are possible) to a material, such as the substrate, for solid state culture described herein. Steaming is one of the common methods of sterilization, for the elimination of microorganisms such as bacteria. Water vaporizes when heated to 100° C. under standard atmosphere pressure (100 kPa). However, under higher pressure, water will only vaporize at temperature higher than 100° C. Thus steaming can be carried out at ambient pressure, such as atmosphere pressure, without extra pressure being applied. Alternatively, steaming can be carried out under pressure higher than 100 kPa (generally referred to as “autoclave”). Autoclaves commonly use steam heated to 121° C. (250° F.), at 103 kPa (15 psi) above atmospheric pressure. Solid surfaces are effectively sterilized when heated this temperature for at least 15 minutes or to 134° C. for a minimum of 3 minutes. “Effective sterilization” in this context means to reduce undesired microorganisms, such as bacteria, to the extent that they cannot interfere with the enzyme production process.

Steaming can also be used to adjust the amount of water in the substrate. A certain amount water is necessary for the growth of the fungus. Water can be added to the substrate together with other components. However, because of the limited amount of water needed for making the substrate, it may be difficult to mix the water evenly in the substrate. Thus, steaming, among others, such as sprinkling during mixing, is a convenient way to introduce water to the substrate evenly. Substrate moisture after steaming may be in the range of 30 to 80% preferably 40 to 50% in barley substrates. Final moisture after addition of a liquid inoculum culture is preferably in the range of 45 to 55%. In some embodiments, water, and or nutrient solution and steam combine to produce final substrate moisture of 40 to 80%, preferably 50 to 70%.

Substrate may be decontaminated by tyndalization or double steaming. Substrate is steamed to about 90° C. cooled, held for 1 hour to 24 hours then steamed a second time to about 90° C. The first steaming kills any vegetative cells and induces spores to germinate and grow. The second steaming kills the vegetative cells from spores that survived the first steaming. Moisture added to the substrate is adjusted so that the final moisture after tyndalization is in the ranges described above. If other decontamination techniques are used, water may need to be introduced separately.

Thus, in one aspect of the present invention, the substrate is steamed to adjust moisture and reduce contamination from indigenous microorganisms. This can be carried in an open space, where the substrate is spread out on a surface, such as the floor, or the bottom of a container. However, preferably, steaming is carried out in a contained space, such as a growth chamber, and optionally, mechanical components are used to move the substrate to assist the dispersion of the steam. Steaming can carried out under pressures higher than atmosphere pressure when steam is introduced into a closed, pressured system. Alternatively, steaming is carried out at ambient pressure, such as the same as the atmosphere pressure, where the steam is introduced into an open system.

The way to generate steam is known in the art, as well as the way of steaming. The duration of the steaming depends on the amount and density of the substrate and temperature and pressure of the steam. It can be from several minutes to several hours, preferably from 5 minutes to one hour, or preferably for 15 to 30 minutes.

After steaming, the substrate will be let cooled down to a temperature suitable for the growth of fungus, either by naturally cooling down over time, or by applying cold air to the substrate.

The substrate then can be used to grow fungus to produce the enzyme preparation of the invention.

In another aspect of the invention, substrate is prepared and microbial contamination reduced by extrusion. Extruders such as those used to produce pasta or pelleted livestock feeds force a material through a small opening in a die where mechanical force creates high temperature and pressure. In the present invention, substrate ingredients are blended, wetted with nutrient solution and extruded through a die to form a pellet. Substrate may be heated in the extruder barrel by steaming or other means to a temperature of 70 to 150° C. prior to being forced through the die. Extruders may be of a single or twin screw design. Temperature in the die ranges from 70 to 200° C. preferably about 150° C. and pressure from 100 to 400 psi, preferably about 300 psi. The high pressure and temperature kills contaminating microbes and forms the substrate into a pellet which has good physical characteristics in solid culture.

In one embodiment, the substrate used for the SSC cellulase enzyme preparation is a combination of cellulose containing compounds, primarily a fraction of corn cob and straw supplemented with barley and wheat germ.

In some embodiments the substrate contains 30 to 80% of BPC derived from corn cob, preferably about 45%; Barley flour 10 to 60% preferably 40%; milled grain straw 1 to 5 mm particle size 1 to 15% preferably about 5%, and wheat germ 1 to 20% preferably about 10%. In some embodiments whole ground barley or barley pearling waste may substitute for barley flour. In some embodiments straw may be grass seed or other straw and may be milled to a fine powder up to 10 mm average particle size.

B. Fungal Inoculum Preparation, Incubation, and Culture Control

The steamed substrate is inoculated with the desired fungus (the inoculum) and loaded into a growth chamber. The fungus grows on the substrate, utilizing it as a food source and at the same time, producing the desired enzymes.

By “inoculum” or “inoculant” herein is meant the material used in an inoculation. For example, the fungus provided in the present invention, or the fungus that are obtained through the methods provided in the present invention, or any other suitable fungus, is produced in conventional liquid culture known in the art to produce a large volume of cell mass. These cells are sprayed on the steamed substrate as an inoculum. In laboratory scale cultures, inoculum is poured onto the prepared substrate and stirred. In larger scale systems, the inoculum is sprayed onto the substrate as the substrate is conveyed into the growth chamber or is sprayed on the substrate as the substrate is mixed. In one embodiment the substrate is steamed in a chamber fitted with mixing. After the substrate is steamed and cooled inoculum is sprayed onto the substrate as the substrate is mixed. In another embodiment, substrate pellets from the extruder cool as it is conveyed and inoculum sprayed onto the pellets as pellets are transferred into the growth chamber.

The methods to produce inoculum are well known in the art. Generally, fungus from a stock can be used to grow, either in a liquid medium or on a solid medium, for a period of time under proper temperature. The stock can be obtained from many sources, such as from American Type Culture Collection (ATCC), or other collections of fungus, such as the Fungal Genetics Stock Center at University of Missouri, Kansas City, or a collection kept in-house. The stock can be in the form of conidia (asexual, non-motile spores of a fungus) stored in silica gel or in lyophilized, or as non-spore form kept in a suitable medium for preservation. Any medium that is suitable for the growth of the fungus can be used. The temperature for growing the fungus is 10 to 40° C., preferably 20 to 35° C., and more preferably 30° C. The incubation time is 24 to 96 hours preferably about 48 hours. After the fungus reach the desired density in the liquid culture, or desired colony size on the surface of solid culture media, they are harvested to be used to inoculate substrate in a growth chamber. Either the fungi, the spores formed by the fungi, or mixture of both, can be used as inoculum. Spores can be harvested by methods known in the art, for example, by washing the surface of a agar plate on which fungi grown with either water or buffer, and separate spores by known methods, such as filtering and centrifugation. Spores are easy to store and have a much longer shelf life.

Among the factors that determine morphology and the general course of fungal fermentations, the type and size of inoculum is of prime importance. The preferred embodiment employs a liquid culture inoculum. By “inoculum size” herein is meant the amount of inoculum being used for the inoculation. It is generally measured by the percentage of inoculum weight over the substrate weight. Suitable type and size of inoculum can be determined using methods known in the art. For example, different inoculum size, such as from 0.1 to 20%, can be tested in small scale fermentor to determine the optimal size. Inoculum size can be from 1 to 10%, preferably is about 2-5%.

In one aspect, the present invention provides methods of incubating and growing fungi in a growth chamber or bioreactor using solid state culture technique. The preferred embodiment employs a liquid culture inoculum.

By “growth chamber” or “bioreactor” herein is meant any device or system that supports a biologically active environment, particularly a device capable of holding moist solid fermentation media inoculated with microorganism and carrying out the process of solid state fermentation in a contained manner. A growth chamber can be used to grow any microorganism capable of growing under specified conditions in a contained environment. The bioreactor's environmental conditions like air composition, gas (i.e., air, oxygen, nitrogen, carbon dioxide) flow rates, temperature, pH, humidity, intensity of light, and dissolved oxygen levels, and agitation speed/circulation rate can to be closely monitored and controlled to provide a desired environment for the microorganisms to grow. Bioreactors can be of any size and shape and any configuration that will physically hold the solid substrate in which growth conditions can be maintained. A number of reactor configurations have been tested for solid substrate culture for cellulase enzyme production including columns, cylinders with supporting trays.

In some embodiments, the growth chambers are rectangular in shape and constructed of mild steel or plastic panels designed for ease in cleaning. For example growth chambers designed for commercial use might have dimensions of 10 feet wide, 10 feet high and 60 feet long with a series of trays or shelves stacked at 6 inch to one foot intervals. Shelves are constructed of metal mesh to allow air circulation from the bottom. The growth chamber has doors at both ends of the rectangle. To load the growth chamber inoculated solid substrate is fed onto a flexible net at the “loading end door” as the net is pulled across the support shelf (from the door at the opposite end). When the culture net reaches the length of the growth chamber the net pull is stopped. This is repeated for each shelf. When all shelves are full, the culture incubation proceeds for the desired time. At the conclusion of the culture period the net on each shelf is pulled from the growth chamber at through the “unloading end door”. The fungal culture is scraped from the net onto a conveyor for transport to drying or other processing.) The net system allows a small number of people to efficiently load and unload tons of solid substrate culture. Bioreactor makers use vessels, sensors, controllers, and a control system, networked together for their bioreactor system. Fouling (the accumulation and deposition of living organisms and certain non-living material on hard surfaces in an aquatic environment) can harm the overall sterility and efficiency of the bioreactor, especially the heat exchangers. To avoid it the bioreactor preferably is easily cleanable and is as smooth as possible. Biological fermentation is a major source of heat. A flow of temperature controlled air is used to maintain temperature at optimum for fungal growth.

Generally, it is preferred to control the conditions inside the growth chamber throughout the incubation period. The conditions include, but are not limited to, temperature, humidity, pressure, air composition (such as oxygen, carbon dioxide, and nitrogen concentration), pH, and air circulation status. Thus, in many embodiments, the growth chamber preferably is attached to a variety of sensors to monitor the conditions, such as temperature, humidity, pressure, air composition, and pH within the chamber. A variety of sensors are known in the art that can be used to monitor the conditions within the growth chamber. In one embodiment, standard sensors known to the art are used to measure temperature in the substrate and in air, humidity, oxygen concentration in the air, and air flow rate. Thus, during the incubation, one or more parameters can be monitored and/or controlled.

In one embodiment, a Programmable Logic Controller®, PLC®, or Programmable Controller is used to control the reactor. A programmable controller is an electronic device used for automation of industrial processes, such as control of machinery on factory assembly lines. Unlike general-purpose computers, the PLC is designed for multiple inputs and output arrangements, extended temperature ranges, immunity to electrical noise, and resistance to vibration and impact. Programs to control machine operation are typically stored in battery-backed or non-volatile memory. A PLC is a real time system where output result is produced in response to input conditions within a bounded time.

PLC generally has extensive input/output (I/O) arrangements. These connect the PLC to sensors and actuators. PLCs read limit switches, analog process variables (such as temperature and pressure), and the positions of complex positioning systems. On the actuator side, PLCs operate electric motors, pneumatic or hydraulic cylinders, magnetic relays or solenoids, or analog outputs. The input/output arrangements may be built into a simple PLC, or the PLC may have external I/O modules attached to a computer network that plugs into the PLC. PLCs may also have a human-machine interface to interact with people for the purpose of configuration, alarm reporting or everyday control.

In some embodiments, the present invention uses a process control program and PC to monitor pretty simple feedback loops to control Solid Substrate Culture incubation of the fungi. The computer controls electronically actuated valves opened or closed to provide: outside air (or tank gas flow) to responding to temperature measurement and oxygen concentration in chamber atmosphere to control oxygen level; and steam injection into the air flow in response to humidity measurement. Control systems may also divert air flow through heaters or refrigeration to heat or cool the air circulating through the growth chamber.

One property for monitoring and control is temperature. Controlling the temperature of large quantities of rapidly growing fungal culture is preferred in some embodiments. The growth rate of fungus can depend on the temperature. In general, the growth of fungus is a heat generating process, cooling is more likely to be used than heating. If not controlled or removed, metabolic heat generation can increase culture bed temperature to the point where fungal growth is inhibited.

The control of temperature is by transfer of heat in or out of the growth chamber, thus heating or cooling the temperature inside the growth chamber. There are a variety of methods to transfer heat and control the temperature. In some embodiments, the control of the temperature is by circulation of air. For example, circulation of air inside the growth chamber can be coupled with the exchange of the air between the inside and the outside of the chamber, Alternatively, hot or cold air can be blown into the chamber if desired.

In some embodiments, a thermal jacket can be attached to the outside of the chamber, with heat carrying media inside the thermal jacket. The heat carrying media can be solid material or aqueous liquid, such as water, circulation in it. The liquid can be cold or warm, depends on whether cooling or heating is desired. The thermal jacket can be connected to a heating or cooling device. Alternatively, the thermal jacket can comprise a cooling or heating device itself. In some embodiment, the thermal jacket comprises an electric heater. In some embodiments, trays that support culture media may incorporate temperature control by means of circulating a fluid (water) through the tray.

In many embodiments, a constant or substantially constant temperature is maintained inside the growth chamber. This can be accomplished by methods such as agitation of the substrate.

In some embodiment, the transfer of heat between the growth chamber and outside is combined with the agitation of the substrate inside the chamber to maintain a substantially constant temperature inside the growth chamber.

The temperature inside the growth chamber should be controlled for optimal fungus production. It is between from 10 to 50° C., preferably from 20 to 40° C. and even more preferably from 25 to 32° C.

Usually, during the incubation, the fungus metabolizes the substrate, and generates heat (in addition to enzymes). Waste heat is generally low value heat, typically less than 30° C. It can be used as supplemental room heat or exhausted to atmosphere.

The air composition within the chamber is also important. Fungus grows aerobically, thus, sufficient supply of oxygen is important. Carbon dioxide is generated by the fungus, and should be removed from the chamber from time to time to prevent the inhibition of fungus growth. Thus a good air circulation system is provided in many embodiments. Fresh air (for example, from the environment), or gas (in controlled ratio), can be introduced into the chamber to replace the air therein. Generally, air from the atmosphere contains 78% nitrogen, 20.95% oxygen, 0.93% 0.04% carbon dioxide, and about 1% water vapor.

The growth chamber generally also includes at least an inlet and an outlet to allow the circulation of air (or gas) inside the growth chamber. The air come into the chamber is preferably pre-cleaned, such as by filtering, to remove undesired contaminants, particularly bacteria. The source of the air can be from the atmosphere, or from a gas tank with a pre-mixture of gas, including, but not limited to oxygen, nitrogen, and carbon dioxide. Alternatively, gas, such as oxygen, can be pre-mixed with air, and is injected into the growth chamber through an optionally separate inlet. Steam can also be introduced into the growth chamber if desired to maintain the humidity inside the growth chamber. In some case the outlet is connected to a cleaning component, such as a filter, to prevent the released air to contaminate the environment. For example, it can be important to prevent the spores that may be generated during culture from being released from the growth chamber.

The pressure inside the chamber is generally the same at the atmosphere at the site. However, it may be desirable to have pressures lower or higher the atmosphere at the site. For example, the pressure inside the chamber may be lower than outside in order to prevent the spores produced during the incubation from escaping to contaminate the environment. Conversely, the pressure inside the chamber may be higher than outside to prevent microorganisms, such as bacteria, from entering the growth chamber. Generally growth chamber is maintained at positive pressure to prevent introduction of contaminants.

For optimal fungus growth, generally, the oxygen within the chamber is from 15 to 21%, and even more preferably is about 21% as in air. The concentration of carbon dioxide is maintained at normal atmosphere air concentration of about 450 ppm.

The humidity inside the growth chamber is also controlled. Generally, humidity is measured in term of relative humidity (“RH”), which is defined as the ratio of the partial pressure of water vapor in a gaseous mixture of air and water to the saturated vapor pressure of water at a given temperature. During culture incubation, the RH inside the chamber is from 70 to 100% (w/w), preferably from 80 to 100%, and even more preferably from 90 to 95%.

Generally, the substrate pH is adjusted to about pH 5 with addition of mono basic potassium phosphate in the nutrient solution used to wet the substrate. The pH generally is not adjusted during incubation.

The process uses technology innovations that are adapted from the malting and mushroom industries. The mechanics of being able to move large quantities of solid substrate, mix and maintain uniform moisture, and uniformly heat and cool the beds is known in the art. In one aspect, a mixing component is employed to move and mix the substrate within the growth chamber. The mixing component includes, but is not limited to, blades that can blend the substrate, a shaking device on top of which the substrate is placed, a tumbling device, or a device that can move the growth chamber, such as rotating it.

The incubation time varies depending on the enzymes being produced. It is from 3 to 20 days, preferably 5 to 10 days, and more preferably about seven days for growing Trichoderma in solid culture for enzyme production. After the incubation, the whole culture is harvested for next step.

In one embodiment, the fungi metabolize approximately 35% of the substrate during incubation. It exits the growth chamber at 55% moisture. Every 100 lbs of substrate input will result in 65 lbs of enzyme preparation out. On a dry weight basis substrate utilization ranges from about 10% to about 40%, typically about 20%.

C. Enzyme Preparation

The present invention provides a cellulase/hemicellulase enzyme preparation and methods of making the cellulase/hemicellulase enzyme preparation. The usage of the enzyme preparation as provided in the present invention provides significant cost-reduction in producing ethanol from cellulose.

By “enzyme preparation” herein is meant the composition containing a mixture of enzymes that efficiently hydrolyze cellulose and hemicellulose under conditions suitable for fermentation. By “cellulase/hemicellulase enzyme preparation” or “SSC ellulase” herein is meant the enzyme mix that comprises cellulase and hemicellulase prepared with the SSC process provided herein. The enzyme activities can be measured by methods known in the art, for example, the filter paper method described herein. The table in examples shows representative enzyme composition.

There is commercially available enzyme mixtures for cellulose hydrolysis. For example, Accelase™ 100 enzyme complex (Genencor) contains multiple enzyme activities: exoglucase, endoglucanase, hemi-cellulase and beta-glucosidase. It is produced with a genetically modified strain derived from Trichoderma reesei.

One of the surprising advantages of the SSC cellulase provided herein is that it could be produced with fungus that has not been genetically modified.

In a preferred embodiment, the SSC cellulase is prepared from a fungus that has not been genetically engineered. Thus, the multiple enzyme activities contained in the SSC cellulase are from the process for growing the fungus as provided herein, rather than using a genetic engineered strain of a fungus. In some embodiments, the SSC celluase is not produced from a genetically modified strain of Trichoderma reesei.

By “low pH” herein is meant the pH from 4 to 7, preferably from 4.5 to 5.0.

By “ambient temperature” herein is meant a temperature between 20 to 40° C. Preferably the temperature is 30 to 35° C.

In many embodiments, the whole culture is used as an enzyme preparation without any purification steps. This way, the cost of producing enzyme preparation can be dramatically reduced. Accordingly, the whole culture is slurried and pumped to an ethanol fermentation tank or dried and stored for future use. Since the whole culture is used as the enzyme preparation, there is no significant waste product to dispose of.

If the enzyme preparation is to be used directly in the ethanol production process, water can be added to the whole culture to make a slurry. The amount of water to be added depends on the water content of the whole culture, but is an amount that makes the slurry easily pumped. For example the slurry would be 1 to 10% solids.

The enzyme preparation can be used in the ethanol production methods provided herein or any other suitable process known in the art. For example, it can be used in the process described in U.S. Pat. No. 5,348,871, the entire disclosure of which is incorporated by reference.

Alternatively, the whole culture can be dried for storage using methods known in the art. Cultures are dried using a flow of warm dry air at a temperature of about 20 to 50° C.

In many embodiments, the whole culture is harvested for purifying enzymes that can be used to convert cellulose to sugar. The purification can be carried out according methods known in the art to separate the enzyme proteins from the culture substrate, for example by extracting the culture in water or buffer solution (e.g. ultrafiltration and/or diafiltration) then concentrating the resulting enzyme containing solution or by using known chromatography techniques to purify the enzyme proteins. The purification can be complete or partial.

Generally, the fungi are harvested and separated from the culture media by methods known in the art, such as mixing the culture material in water or buffer solution and centrifugation to separate the liquid fraction containing the enzyme from the residual culture solids. Optionally, the fungi are washed with water or buffer solution, preferably cold, for several times.

To prevent enzyme degradation and denaturing, the purification is preferably carried at low temperature, such as at 4° C., and in the presence proteinase inhibitors. There are many proteinase inhibitors known in the art and are commercially available.

After the enzymes are released from the cell, a variety of methods known in the art can be used for purification. Standard purification methods include chromatographic techniques, such as ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation (such as ammonium sulfate precipitation), dialysis, and chromatofocusing techniques. Ultrafiltration and diafiltration techniques, in conjunction with protein concentration, are also useful.

The amount of SSC Cellulase enzyme preparation required for cellulose hydrolysis depends on the enzyme activities of the enzyme preparation as well as the nature of the feed stock that provides the source of the cellulose. Thus, the enzyme activities of the enzyme preparation and the content of the cellulose of the feedstock can be measured using methods known in the art or those described herein. Small scale pilot runs can also be conducted to determine the optimal amount of enzyme preparation needed.

In some embodiments, the SSC Cellulase enzyme preparation is typically added to the ethanol fermentation at about 0.5 to 30% % w/w whole culture enzyme preparation to feedstock. In the SSC system, data expresses enzyme loading as weight percent of dry whole SSC culture to dry feedstock input. Because the process design generally employs whole culture material without any separation or purification, enzyme preparations contain cell mass and residual substrate including protein in substrate components. The whole crude enzyme preparation is used on a weight basis in the hydrolysis process.

The enzyme activities contained in the preparations provided herein are defined by selective substrate enzyme assays and found to generally include endo and exo acting cellulases, cellobiase, and xylanases. Hydrolysis of cellulose was determined in assays measuring the conversion of filter paper to glucose. Optimal cellulose hydrolysis activity is at pH 4.5 to 5.0. Cellulose hydrolysis occurs at 20 to 50° C. Enzyme preparations produced by the methods provided in the present invention were compared to commercial cellulase preparations. The SSC produced cellulases contained less filter paper units per gram than did the commercial cellulases. However in assays comparing hydrolysis of delignified straw, SSC cellulases were superior to commercial cellulases on an equal weight basis.

In one aspect, the enzyme composition provided by the present invention comprises cellulases. By “cellulase (E.C. 3.2.1.4)” herein is meant enzymes that catalyze the cellulolysis (or hydrolysis of cellulose). Cellulases are produced chiefly by fungi, bacteria, and protozoans. There are also cellulases produced by other types of organisms such as plants and animals. Several different kinds of cellulases are known, which differ structurally and mechanistically. There are five general types of cellulases based on the type of reaction catalyzed. They are endo-cellulases, exo-cellulases, cellobiases, oxidative cellulases, and cellulose phosphorylases.

Thus in one aspect, the enzyme preparation comprises endo-cellulase. Endo-cellulase breaks internal bonds to disrupt the crystalline structure of cellulose and expose individual cellulose polysaccharide chains.

In one aspect, the enzyme preparation comprises exo-cellulase. Exo-cellulase cleaves 2-4 units from the ends of the exposed chains produced by endocellulase, resulting in the tetrasaccharides or disaccharide such as cellobiose. There are two main types of exo-cellulases (or cellobiohydrolases): one type working processively from the reducing end, and one type working processively from the non-reducing end of cellulose.

In another aspect, the enzyme preparation comprises cellobiase. Cellobiase (or beta-glucosidase) hydrolyzes the endo-cellulase product into individual monosaccharides.

Methods for measuring the activities of endo-cellulase, exo-cellulase, and cellobiase are known in the art. See e.g., Howard G. T. and Elliot L. P., Effects of Cellulolytic Ruminal Bacteria and of Cell Extracts on Germination of Euonymus americanus L. Seeds, Applied and Environmental Microbiology, 54(1):218-224 (1988), herein incorporated by reference.

In yet another aspect, the enzyme preparation comprises hemicellulase. In some embodiments, the hemicellulase is xylanse. Xylanase is a class of enzymes that degrade the linear polysaccharide beta-1,4-xylan into xylose, thus breaking down hemicellulose, which is a major component of the cell wall of plants.

Methods for measuring the activities of endo-cellulase, exo-cellulase, and cellobiase are known in the art. See e.g., EP 1433844, herein incorporated by reference.

Additional enzymes may be added in the compositions and methods encompassed by the invention. For example, cellobiose, an intermediate disaccharide formed according to equation (3), inhibits the hydrolysis reaction. Therefore, it there is insufficient amount of cellobiase in the enzyme preparation, extra amount of cellobiase can be added to increase the hydrolysis efficiency.

In some embodiments, extra amounts of endo-cellulase, exo-cellulase, cellobiase and/or xylanses can be added to the enzyme preparation for better conversion of cellulose to sugar. These enzymes can be obtained commercially from Genencor, logen, and Novozyme, or through methods known in the art. For example, high concentration cellulase/xylanase complex and beta glucanase/xylanase complex are marketed by Genencor International Inc.

The effective amount of these enzymes to be included in the methods of the invention can be readily determined by one skilled in the art.

### Ill. Ethanol Production Process

There has been a lot of interest in conversion of cellulosic biomass to ethanol. Abundant and cheap cellulosic biomass coupled with a growing demand for ethanol is fueling this interest. Stephanopoulos, G., Challenges in Engineering Microbes for Biofuels Production, Science, 315: 801-804 (2007); Himmel., M. E., Biomass Recalcitrance: Engineering Plants and Enzymes for Biofuels Production, Science, 315:804-807 (2007).

Generally, in biomass-to-biofuels (B2B) process, after harvest, biomass is reduced in size and then treated to loosen up the lignin-cellulose fiber entanglement in a step that can take from a few minutes to many hours. Several methods can be used for this purpose, such as biomass treatment with saturated steam at 200° C., explosion with ammonia, and cooking with warm dilute acid or heating with dilute alkali solution. Dilute acid pretreatments are fast (minutes), and hydrolyze some or all of the hemicellulose depending on process conditions. Dilute alkali pretreatments may take from a few minutes to several hours. Steam-based treatments can take up to a day. Alkali and steam treatments typically do not hydrolyze hemicellulose. After pretreatment, the solid suspension is exposed to cellulolytic hemicellulolytic enzymes that digest the cellulosic and hemicellulosic biomass components to release the hydrolysis products, primarily six- and five-carbon sugars, respectively (along with acetic acid and lignin-derived phenolic by-products). The type of pretreatment defines the optimal enzyme mixture to be used and the composition of the hydrolysis products. The latter are fermented by ethanol-producing microorganisms, naturally occurred, selected, or genetically engineered, including but not limited to, yeasts such as Saccaromyces cerevisiae or Pichia stipitis, or bacteria such as Zymomonas mobilis, Escherichia coli.

In one aspect of the present invention, cellulose hydrolysis and fermentation are combined in a single unit, termed Simultaneous Saccharification Fermentation (SSF) stage. The rationale of combining saccharification (the breaking up of complex carbohydrates into monosaccharides) and fermentation (the conversion of a carbohydrate to carbon dioxide and alcohol) in a single unit is to prevent inhibition of the hydrolytic enzymes by the reaction products. The SSF step typically lasts 3 to 6 days, with cellulose hydrolysis the slow, limiting step. The product of SSF is a relatively dilute ethanol stream of 2 to 4.5% from which ethanol is separated by distillation.

A. Cellulose Hydrolysis

The enzyme preparation provided herein can be used in either separate or simultaneous hydrolysis or fermentation. Enzymatic hydrolysis and fermentation processes of hemicellulose and hemicellulose are described in the literature. However, the enzyme preparation provided herein has the particular advantage of allowing a process design in which the lingocellulose can be pretreated with mild process conditions that do not hydrolyze hemicellulose. Cellulose and hemicellulose can then be enzymatically hydrolyzed in a single process step.

By “cellulosic biomass” or “cellulosic feedstock” herein is meant materials that contain cellulose. It includes, but is not limited to, wood or wood waste, straw, herbaceous crops, corn stover, grass such as switch grass, or other sources of annual or perennial grass, or any delignified cellulose such as paper or paper waste, pulp and paper mill waste, municipal and industrial solid wastes.

The cellulosic feedstock primarily consists of cellulose, hemicellulose, and lignin bound together in a complex structure along with small quantities of extractives, pectins, proteins, and ash. Due to the complex chemical structure of the cellulosic feedstock, microorganisms and enzymes cannot effectively attack the cellulose without prior treatment because the cellulose is highly inaccessible to enzymes or bacteria. This inaccessibility is illustrated by the inability of cattle to digest wood with its high lignin content even though they can digest cellulose from such material as grass. Successful commercial use of biomass as a chemical feedstock depends on the separation of lignin from cellulose, hemicellulose and from other constituents.

In general, as described below, cellulosic feedstock is grounded or processed to reduce the particle size and/or increase surface/volume ratio.

By “cellulose” herein is meant a polysaccharide of beta-glucose that has the formula (C₅H₁₀O₅)ₙ. Cellulose forms the primary structural component of green plants. The primary cell wall of green plants is made of cellulose; the secondary wall contains cellulose with variable amounts of lignin.

Cellulosic feedstock in an untreated or minimally treated form is referred to as “raw feedstock”; treatments as outlined herein results in the “cellulosic feedstock” in the saccharification process.

The amount of cellulose can be measured by methods known in the art. For example, Updegraff D M, Semimicro Determination of Cellulose in Biological Materials, Analytical Biochemistry 32: 420-424 (1969) herein incorporated by reference.

Many cellulosic feedstocks contain lignin. Lignin and cellulose, considered together, are termed lignocellulose; lignocellulosic feedstocks can also be used in the present invention. It is noted that cellulosic feedstocks and lignocellulosic feedstocks are not mutual exclusive terms. One of the abundant lingocellulosic feedstock is sugarcane residue, called bagasse, is generated during the milling of sugarcane and is plentiful in tropical and subtropical regions. Other lignocellulosic feedstocks include, but not limited to, agricultural residues such as corn stover, wheat and rice straw, and forestry residue; industrial residue such as pulp and paper processing waste; and energy crops such as switchgrass. Unlike starch which contains homogenous and easily hydrolyzed polymers, lignocellulose plant matter contains cellulose (23-53%), hemicellulose (20-35%), polyphenolic lignin (10-25%) and other extractable components. Knauf M., and Monirussaman M., Ligocellulosic biomass processing. A perspective, International Sugar Journal, 106(1263); 147-150 (2004), herein incorporated by reference.

In one aspect, the present invention provides using SSC cellulase to hydrolyze cellulosic feedstock with lignin. In some embodiments, the cellulosic feedstock may be pretreated to remove or disassociate the lignin and make the cellulose accessible to cellulase enzymes. Processes to convert cellulosic feedstock into ethanol have a number of features in common: (1) particle size reduction; (2) disassociation of lignin and cellulose; (3) conversion of cellulose to sugar; and (4) fermentation of sugar to produce ethanol.

A fine grind exposes more surface area of the cellulosic feedstock, and can facilitate saccharification and fermentation. The cellulosic feedstock can be reduced in size by a variety of methods, e.g., by grinding, to make the cellulose more available for hydrolysis and fermentation. Other methods of reducing the size of cellulosic feedstock are available. For example, vegetable material, such as straw, can be ground using mills (ball, mill, hammer, etc.), and/or other methods for the purposes of particle size reduction. Also can be used is emulsion technology, rotary pulsation, sonication, magnetostriction, ferromagnetic materials, or the like. These methods of cellulosic feedstock size reduction can be employed for substrate pretreatment. Without being bound by theory, it is believed that these methods increase the surface area of the cellulosic feedstock while raising the effectiveness of flowing of liquefied media (i.e. decreased viscosity). It is also believed that the initial size reduction is required to increase the amount of biomass surface area that can be contacted with acid, solvent, steam, enzymes or chemicals that might be used to disassociate the lignin from the cellulose and hemicellulose. These methods can include mechanical, and sound based vibrations at varying speeds. This can provide varying frequencies over a wide range of frequencies, which can be effective for pre-treating the cellulosic feedstock and/or reducing particle size. In some embodiments, particle size of feedstocks is reduced as a first step. For straw a hammer mill is used. Also can be used are air swept pulverizer which reduces lignocellulosic material to a flour consistency of very small particle size. Any size reduction generally will work.

Different methods known in the art can be used to remove lignin from the cellulosic material. In some embodiments, an alkaline pretreatment process is used to remove lignin from cellulosic material, such as straw. The alkaline pretreatment does not degrade the hemicellulose found in cellulose material. Hemicellulose is a polymeric structure composed primarily of five carbon sugars such as xylose. The hemicellulose can be conserved for potential conversion to ethanol (although this requires different fermentation organisms as commercial distillery yeast do not ferment five carbon sugars to ethanol). With the current technology, most of the hemicellulose is carried through with residual non-cellulose solids.

In some embodiments, a process known as “steam explosion” developed by Canadian company SunOpta Inc is used to pre-treat the cellulosic feedstock. See also U.S. Pat. No. 5,769,934, herein incorporated by reference.

In another aspect, the SSC Cellulase provided herein can be used to produce ethanol from transgenic plants. For example, some embodiments use transgenic plants carry extra or exogenous genes for protease, lignase, cellulase, or both. U.S. Patent Application Publication No. 20060185037 describes transgenic plants containing ligninase and cellulase which degrade lignin and cellulose to fermentable sugars, and herein is incorporated in its entirety. It discloses transgenic plants comprise ligninase and cellulase genes from microbes operably linked to a DNA encoding a signal peptide which targets the fusion polypeptide produced therefrom to an organelle of the plant, in particular the chloroplasts. When the transgenic plants are harvested, the plants are ground to release the ligninase and cellulase which then degrade the lignin and cellulose of the transgenic plants. Alternatively, transgenic plants can be generated to carry only lignase genes. In either case, after delingification, the plants can be used in the process provided by the present invention to produce ethanol.

In one aspect, the present invention provides using SSC cellulase to hydrolize material with low levels of or no lignin. There are a variety of cellulose source materials that have low level or lignin, such as waste paper, paper mill waste, cotton gin trash cotton lint waste.

In one aspect, the present invention provides processes that uses the solid substrate culture cellulase provided herein used in an enzymatic process to convert pretreated lignocellulose to fermentable sugars. The process could be sequential hydrolysis and fermentation, simultaneous hydrolysis and fermentation, in batch, or be continuous. One advantage of the SSC cellulase provided herein is to provide multiple activities, hydrolyzing hemicellulose and cellulose in a single step. In addition, the SSC cellulase provided herein is also effective under a wide range of commercial conditions, for example with pretreated, pH adjusted but unwashed lignocellulose as shown in Example 6.

By “hydrolysis”, “cellulolysis”, “saccharification” or “saccharifying” herein is meant the process of converting cellulose to smaller polysaccharides and eventually to monosaccharides, such as glucose, with enzymes, e.g., cellulase, and hemicellulose to smaller polysaccharides such as xylose.

A saccharifying enzyme composition can include any of a variety of known enzymes suitable for converting cellulosic feedstock to fermentable sugars, such as cellulase (e.g., endo-cellulase and/or exo-celluase).

In one embodiment, saccharifying can include mixing the cellulosic feedstock with a liquid, such as water, which can form a slurry or suspension and adding the enzyme preparations of the present invention to the liquid. Alternatively, the addition of the enzyme preparation can precede or occur simultaneously with mixing. The cellulosic feedstock can be mixed with liquid at about 1 to 20% with the preferred range between 5 and 15% solids for pretreated lignocellulose. Viscosity of pretreated cellulose in water limits the concentration range of the slurry. Fed batch systems can be used in which additional pretreated lignocellulose is added to the slurry after enzymatic hydrolysis of some of the feedstock has reduced the viscosity. As used herein, wt-% of cellulosic material in a liquid refers to the percentage of dry substance cellulosic material or dry solids.

Suitable liquids include water (e.g. tap water, well water, etc.) and process waters, and mixtures thereof. By “process waters” here is meant the water that has been used in an industrial process, such as stillage (backset), scrubber water, evaporator condensate or distillate, side stripper water from distillation, or other ethanol plant process waters. By “stillage” herein is meant the residue particles and liquid effluent remaining after distillation. In one embodiment, the liquid includes water.

The initial pH of the saccharification mixture can be adjusted as described herein. The pH can be from 4 to 7, preferably from 4 to 6, and more preferably is about 4.5 to 5.0.

The enzyme preparation provided in the present invention can be used as a replacement for expensive purified or partially purified commercially available in conventional multiple-step processes for ethanol production:

B. Fermentation

The sugar from cellulose hydrolysis is subjected to fermentation to produce ethanol. After the saccharification step is completed, yeasts are added to the solution of fermentable sugars to begin fermentation to ethanol. Thus, the steps of cellulose hydrolysis and fermentation can be carried out separately. Alternatively, the steps of cellulose hydrolysis and fermentation can be carried out simultaneously. It is also possible to have overlapping steps of hydrolysis and fermentation. For example, the fermentation step can be initiated after the hydrolysis step starts, but before the hydrolysis is completed. This simultaneous saccharification and fermentation allows for higher concentrations of cellulose to be hydrolyzed and fermented.

The process provided by the present invention includes fermenting sugars from cellulosic feedstock to ethanol. Fermenting can be effected by a microorganism, such as yeast. The fermentation mixture need not, and in an embodiment does not, include protease. However, in some embodiments, the process waters used may contain protease. The amount of protease can be less than that used in the conventional process.

In an embodiment, the present fermentation process produces potable alcohol. Potable alcohol (also known as rectified spirit) is high concentration alcohol purified by the process of rectification (repeated or fractional distillation) has only acceptable, nontoxic levels of other alcohols.

Without being bound by theory, it is believed that known distillery yeast grow well over the pH range of 3 to 6, but are more tolerant of lower pH's down to 3.0 than most contaminant bacterial strains. Contaminating tactic and acetic acid bacteria grow best at pH of 5.0 and above. Thus, in the pH range of 3.0 to 4,5, it is believed that ethanol fermentation will predominate because yeast will grow better than contaminating bacteria. In some embodiments, the present method can include varying the pH. Varying the pH can be done to reduce the likelihood of contamination early in fermentation and/or to increase yeast growth and fermentation during the latter stages of fermentation.

In one embodiment, fermentation is conducted at a pH of about 6 or less, preferably about 4.5 to 5.0. In another embodiment, the present method can include varying the pH. Generally, the pH is adjusted to 4.5 to 5.0 for either separate or simultaneous hydrolysis and fermentation as this is the optimum for the enzyme, and the yeast tolerate a much wider pH than the enzyme.

In some embodiment, the pH is not adjusted during fermentation. The pH can be maintained by adding fresh substrate slurry at the desired pH as described above. The pH is determined by the pH of the components during filling. In one embodiment, the pH is decreased to about 5 or below. In another embodiment, the pH is about pH 4 (e.g. 4.1) at the start of fermentation fill and is increased to about pH 5 (e.g. 5.2) toward the end of fermentation fill. In yet another embodiment, the method includes stopping pH control of the mash slurry after the yeast culture becomes established during the initial process of filling the fermentor, and then allowing the pH to drift during the end stages of filling the fermentor.

One of the important concerns with fermentation systems is the difficulty of maintaining a sterile condition free from bacteria in the large-sized batches and with the long fermentation period. Unfortunately, the optimum atmosphere for fermentation is also extremely conducive to bacterial growth. Should a batch become contaminated, not only must the yeast and sugar solution be discarded, but the entire fermentation vessel must be emptied, cleaned, and sterilized. Such an occurrence is both time-consuming and very costly.

In some embodiments, a variety of methods are used to kill unwanted microorganisms. The methods include, but not limited to, introduction of foreign agents, such as antibiotics, heat, and strong chemical disinfectants, to the fermentation before or during production of ethanol.

Not being by theory, it is believed that higher temperatures early during saccharification and fermentation can increase conversion of cellulose to fermentable sugar when ethanol concentrations are low. This can aid in increasing ethanol yield. At higher ethanol concentrations, this alcohol can adversely affect the yeast. Thus, lower temperatures later during saccharification and fermentation are beneficial to decrease stress on the yeast. This can aid in increasing ethanol yield. Also not being bound by theory, it is believed that higher temperatures early during saccharification and fermentation can reduce viscosity during at least a portion of the fermentation. This can aid in temperature control. Lower temperatures later during saccharification and fermentation are beneficial to reduce the formation of glucose after the yeast has stopped fermenting. Glucose formation late in fermentation can be detrimental to the color of the distillers dried grain co-product.

Generally, in separate hydrolysis the hydrolysis temperature is 20 up to 45° C., where the higher temperature accelerates hydrolysis rate. In simultaneous hydrolysis and fermentation the temperature is determined by the maximum temperature tolerance of the yeast and is maintained at 30 to 35° C. In some embodiments, separate hydrolysis is run at 35 to 45° C., and simultaneous hydrolysis and fermentations are held at a constant 35° C., the typical upper temperature tolerance of yeast.

In some embodiments, the temperature is kept constant during fermentation, thus eliminate the costs of heating and cooling. It is also noted that due to the heat generated by the fermentation process itself, the temperature may shift nevertheless. Thus keeping the fermentation temperature constant should be understood as a relative term, refers to a process without actively shifting the temperature by external heating and cooling.

Any of a variety of yeasts can be employed in the present process. Suitable yeasts include any of a variety of commercially available yeasts, such as commercial strains of Saccharomyces cerevisiae. Suitable strains include “Fali” (Fleischmann's), Thermosac (Alltech), Ethanol Red (LeSafre), BioFerm AFT (North American Bioproducts), and the like. In some embodiments, the yeast is selected to provide rapid growth and fermentation rates in the presence of ambient temperature and medium ethanol levels. In general ethanol tolerance of the yeast is not an issue as the low initial cellulose slurry concentration results in a fermented beer containing at most 5 to 7% ethanol. Other suitable fermentative organisms are those that co ferment both five carbon and six carbon sugars, that is the sugars derived from hemicellulose as well as cellulose. Such organisms include the yeast Pichia stipitis, and Pacchysolen tennophilus.

The ethanol produced during fermentation is inhibitory to yeast due to its osmolality and toxic effects. Yeast is a small microorganism which uses the sugar in the solution as food, and in doing so, expels ethanol and carbon dioxide as byproducts. The carbon dioxide comes off as a gas, bubbling up through the liquid, and the ethanol stays in solution. Generally, the yeast may stagnate when the concentration of the ethanol in solution approaches about 18 percent by volume, whether or not there are still fermentable sugars present. In some embodiments of the present invention, the yeast employed is a recombinant yeast having enhanced stress resistance, such as the yeast strain described in U.S. Pat. No. 5,587,290, or obtained by methods such as the cell evolution method descried in U.S. Pat. No. 7,148,054, herein incorporated by reference. In another embodiment, the recombinant yeast exhibits a modified regulation of the expression of programmed cell death, including senescense.

As an alternative to yeast is Zymomonas mobilis, a bacterium belonging to the genus Zymomonas. See e.g. U.S. Pat. No. 4,443,543, herein incorporated by reference. It is notable for its bioethanol-producing capabilities, which surpass yeast in some aspects. It was originally isolated from alcoholic beverages like the African palm wine, the Mexican pulque, and also as a contaminant of cider and beer in European countries. Compare to yeast, Zymomonas mobilis has higher sugar uptake and ethanol yield, lower biomass production, higher ethanol tolerance.

The amount of yeast starter employed is selected to effectively produce a commercially significant quantity of ethanol in a suitable time, e.g., less than 75 hours. Yeast can be added to the fermentation by any of a variety of methods known for adding yeast to fermentation processes. For example, yeast starter can be added as a dry batch, or by conditioning/propagating. In one embodiment, yeast starter is added as a single inoculation. In an embodiment, yeast is added to the fermentation during the fermenter fill at a rate of 5 to 100 pounds of active dry yeast (ADY) per 100,000 gallons of fermentation mash. In another embodiment, the yeast can be acclimated or conditioned by incubating about 5 to 50 pounds of ADY per 10,000 gallon volume of fermenter volume in a prefermenter or propagation tank. Incubation can be from 8 to 16 hours during the propagation stage, which is also aerated to encourage yeast growth. Typically yeasts are added to an initial cell loading of about one million yeast cells per ml of fermentation.

Another embodiment of the present invention includes the use of a recombinant yeast microorganism having enhanced stress resistance, and exhibiting a modified regulation of the expression of programmed cell death, including senescense. In one embodiment, the recombinant yeast includes a gene or gene fragment that inhibits the expression and/or activity of a polypeptide whose expression is induced by the onset of apoptosis, or that mediates senescence. In another embodiment, the polypeptide that is inhibited is eukaryotic initiation Factor-5A (elF-5A). In one embodiment, the inhibited polypeptide is apoptosis-induced deoxyhypusine synthase (DHS). In another embodiment, the recombinant yeast may include a combination of genes or gene fragments that inhibit the expression and/or activity of more than one polypeptide. In yet another embodiment, the inhibition of the polypeptide results in alteration of the level of senescence. Some embodiments of the present invention include the use of an enhanced fermenting microorganism with technology referenced in U.S. Pat. Nos. 6,878,860, 6,867,237, 6,855,529, 6,849,782, 6,774,284, and 6,538,182, all are incorporated herein by reference in their entirety.

Some embodiments of the present invention use of more than one strain of yeast; for example, complementary and synergistic yeast can be used for fermentation improvements.

In one embodiment, the present method includes solids staging. Solids staging includes filling at a disproportionately higher level of solids during the initial phase of the fermenter fill cycle to increase initial fermentation rates. Solids staging can accelerate enzyme hydrolysis rates and encourage a rapid onset to fermentation by using higher initial fill solids. It is believed that lowering solids in the last half of fill can reduce osmotic pressure related stress effects on the yeast. By maintaining overall fermenter fill solids within a specified range of fermentability, solids staging improves the capacity of the yeast to ferment high gravity mashes toward the end of fermentation. The solids concentration of the mash entering the fermenter can then be decreased as ethanol titers increase and/or as the fermenter fill cycle nears completion.

In some embodiments, nutrient additives that beneficial to the microorganisms used for fermentation (such as yeast) are added to during the fermentation process. In some embodiments, stillage provides nutrients for efficient yeast fermentation, especially free amino nitrogen (FAN) required by yeast.

In some embodiments, the cellulosic feedstock can provide effective fermentation with reduced levels of stillage and even without added stillage. In one embodiment, the present method employs a preparation of cellulosic feedstock that supplies sufficient quantity and quality of nitrogen for efficient fermentation, and no stillage is required, or only low levels of stillage can suffice.

C. Simultaneous Cellulose Hydrolysis and Fermentation

The present process can include simultaneous saccharification and fermentation, using reagents and conditions described above for saccharifying and fermenting. The simultaneous hydrolysis and fermentation generates less heat than a straight fermentation process where all the glucose is made prior to the addition of yeast, thus reduces cooling costs. Generally, in simultaneous hydrolysis and fermentation, yeast consume sugars as they are produced, limiting feedback inhibition of the enzymes. Preferably, the enzyme preparation provided in the present invention is used in the process of simultaneous hydrolysis and fermentation of cellulose described in more details below.

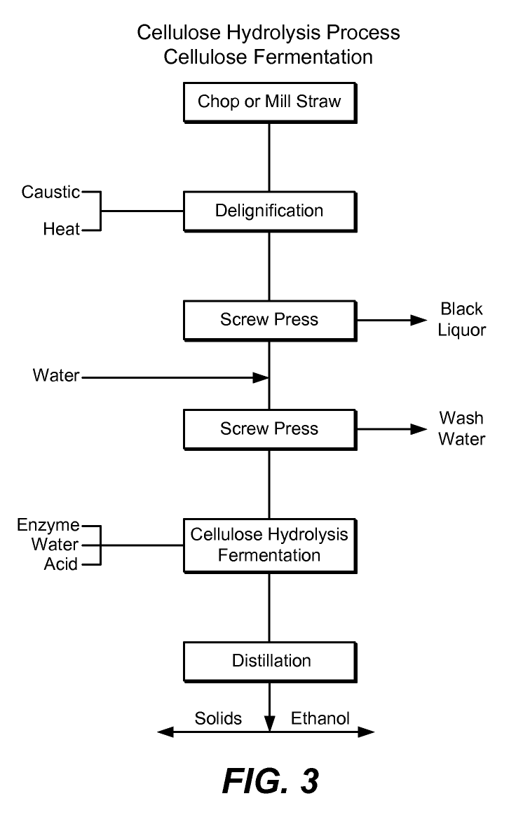

The diagrams shown in FIGS. 2 and 3 provide general schemes of the ethanol production process.

In order for nearly complete fermentation, and in order to produce large quantities of ethanol, the common practice has been to use a batch process wherein extremely large fermentation vessels capable of holding upwards of 500,000 gallons are used. With such large vessels, it is economically unrealistic to provide an amount of yeast sufficient to rapidly ferment the sugar solution. Hence, conventional fermentation processes have required 72 hours and more because such time periods are required for the yeast population to build to the necessary concentration. For example, a quantity of yeast is added to the fermentation vessel. In approximately 45-60 minutes, the yeast population will have doubled; in another 45-60 minutes that new yeast population will have doubled. It takes many hours of such propagation to produce the quantity of yeast necessary to ferment such a large quantity of sugar solution.

The present invention provides an ethanol production process-simultaneous hydrolysis and fermentation of cellulose using SSC cellulase. In this ethanol production process SSC cellulase enzyme and yeast are combined with the mash in a single step in one fermentation vessel with fermentation at fermentation temperature and low pH.

In some embodiments, saccharification and fermentation is conducted at a pH of about 6 or less, and preferably about 4.5 to 5.0. The initial pH of the saccharification and fermentation mixture can be adjusted as described herein. In one embodiment, saccharification and fermentation is conducted for about 24 hours to 7 days, preferably about 48 to 96 hours or more preferably about 72 hours. In an embodiment, the temperature can be decreased as ethanol is produced. For example, in an embodiment, during fermentation the temperature can be as high as about 37° C. and then reduced to about 25° C. This temperature reduction can be coordinated with increased ethanol titers (%) in the fermentor. The preferred temperature range is the maximum tolerated by the yeast, generally about 35° C. (but below 45° C. which is the optimum for the enzyme.)

In one embodiment, simultaneous saccharifying and fermenting can be carried out employing quantities of enzyme preparation and yeast selected for effective fermentation without added exogenous nitrogen; without added protease; and/or without added backset. Backset can be added, if desired, to consume process waters and reduce the amount of wastewater produced by the process.

The amount of enzyme of preparation can be adjusted as to generate optimal output. For example, simultaneous saccharifying and fermenting can employ enzyme preparation at about 1 to 30 and preferably 2.5 to 10% (w/w), of dry solids cellulosic material.

The saccharification and/or fermentation mixture can include additional ingredients to increase the effectiveness of the process. For example, the mixture can include added nutrients (e.g., yeast micronutrients), antibiotics, salts, added enzymes, and the like. Nutrients can be derived from stillage or backset added to the liquid. Suitable salts can include zinc or magnesium salts, such as zinc sulfate, magnesium sulfate, and the like. Extra enzymes can be added, such as protease, phytase, cellulase, hemicellulase, exo- and endo-glucanase, xylanase, and the like.

The concentration of cellulose in the mash and ratio of enzyme to cellulose determines the final concentration rate of ethanol production. Biomass can be up to 40% solids, final ethanol concentration up to 14% v/v and total hydrolysis fermentation time from about 36 to 72 hours. The time is a function of the enzyme and cellulose concentrations. Generally, in practice maximum solids content is limited by viscosity to about 10 to 15% and the maximum ethanol concentration about 3 to at most 7%

The product of the fermentation process is referred to herein as “beer”. For example, fermenting corn produces “corn beer”. Ethanol can be recovered from the fermentation mixture, from the beer, by any of a variety of known processes. For example, ethanol can be recovered by distillation. The remaining stillage includes both liquid and solid material. The liquid and solid can be separated by, for example, centrifugation. The recovered liquid, thin stillage, can be employed as at least part of the liquid for forming the saccharification and fermentation mixture for subsequent batches or runs.

In one embodiment, pretreated cellulose feedstock, enzyme preparation, yeast and water are combined in a single step in one fermentation vessel at approximate ambient temperature. A 30° C. operating temperature helps optimize the performance of the yeast, but the process can operate at lower temperatures. A pH of 4.5 is used to help prevent microbial contamination. The simultaneous hydrolysis and fermentation occur in 24-96 hours, depending on the concentration of cellulose in the slurry, size of the substrate particles and the amount of enzyme added to the mash. The process flow is shown in following diagram in FIG. 2.

The present invention provides the use of SSC cellulase, which is cost effective, in commercial production systems for producing ethanol from cellulose. The SSC cellulase provided by the present invention can be used on waste cellulosic materials that contain no lignin (e.g., waste paper) or low amounts of lignin (Oregon grass straw, switch grass, barley straw, etc.).

In one embodiment, straw is used for the production of ethanol. The process flow used for straw is shown in the following diagram in FIG. 3.

D. Continuous Fermentation

The SSC Cellulase provided herein can be used in a batch or continuous process. A continuous process includes moving (pumping) the saccharifying and/or fermenting mixtures through a series of vessels (e.g., tanks) to provide a sufficient duration for the process. For example, a multiple stage fermentation system can be employed for a continuous process with 48-96 hours residence time. For example, cellulosic material (e.g., fractionated plant material) can be fed into the top of a first vessel for saccharifying and fermenting. Partially incubated and fermented mixture can then be drawn out of the bottom of the first vessel and fed in to the top of a second vessel, and so on.

Without being bound by theory, it is believed that the present method is more suitable than conventional methods for running as a continuous process. The present process should provide reduced opportunity for growth of contaminating organisms in a continuous process. At present, the majority of dry grind ethanol facilities employ batch fermentation technology. This is in part due to the difficulty of preventing losses due to contamination in these conventional processes. For efficient continuous fermentation using traditional technology, the conventional belief is that a separate saccharification stage prior to fermentation is necessary to pre-saccharify the cellulose for fermentation. Such pre-saccharification insures that there is adequate fermentable glucose for the continuous fermentation process.

A continuous stirred tank reactor (CSTR) process overcomes at least some of the limitations of batch processes. The CSTR process features continuous stirring or agitation of the substrate slurry by, for example, mechanical mixing or liquid recycling. The CSTR process allows optimization and balancing of the hydrolysis and fermentation rates to eliminate the large accumulation of glucose and the resulting inhibition of ethanol production. The CSTR process employs continuous addition of fermentable substrate, catalysts and fermentation agents, and continuous removal of any residual substrate—and product—containing broth. The CSTR process has perpetually high concentrations of microorganisms, much reduced down time compared to batch reactors, generally lower maximum concentrations of potentially inhibitory mono- and disaccharides, but higher ethanol concentration. Thus, the relative merits of batch and CSTR will depend upon the needs and circumstances surrounding a given application.

The use of a continuous solids retaining bioreactor (CSRB) provides further improvements in the production of ethanol. The CSRB improves productivity and yield by providing differential solids retention and thus increasing the concentration of substrate particles in the reactor and increasing the hydrolysis rate. The use of a CSRB increases the overall hydrolysis rate and thus reactor productivity by maximizing the amount of cellulose/enzyme complex in the reactor. The key to efficiency in the CSRB process appears to be the management and control of the cellulose/enzyme complex in the reactor.

A further advancement in the production of ethanol is the use of cascaded CSRBs, in which the output from one CSRB reactor vessel becomes the input feed to the next CSRB reactor vessel. This arrangement overcomes the problem of decreased or limited productivity enhancement with high conversion, as the cascaded reactors achieve higher total conversion for an equal cumulative residence time. However, the solids retention in the later stages is always less than in the early stages as a result of reduced cellulose particle size, because smaller particles require more time to settle. An advantage of the cascaded CSRB system over the single CSRB is that at high conversion, the presence of large amounts of ethanol in a single CSRB inhibits the further production of ethanol, whereas this inhibition is alleviated to some extent in a cascade system because the average concentration of alcohol seen by the reaction is reduced as the reaction proceeds through sequential steady state reactors at increasing ethanol concentration until the final concentration is reached.

U.S. Patent Application Publication No. 20060014260 is also incorporated herein by reference. It discloses a semi-continuous simultaneous saccharification and fermentation (SSF) process for the bioconversion of cellulose into ethanol and other organic chemicals

### IV. System for Producing Ethanol

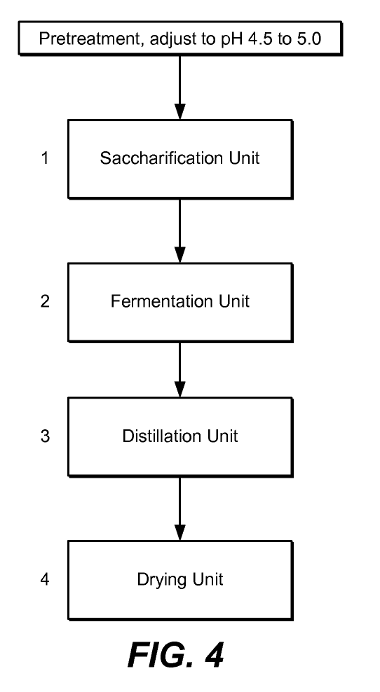

In an embodiment the invention relates to a system that produces ethanol. A diagram of the system in shown in FIG. 4. The present system can include a saccharification unit 1, a fermentation unit 2, a distillation unit 3, and a dryer unit 4.

The saccharification unit 1 can be any of a variety of apparatus suitable for containing or conducting saccharification. The saccharification unit 1 can be, for example, a vessel in which cellulosic material can be converted to a sugar, which can be fermented by a microorganism such as yeast. The saccharification unit 1 can be configured to maintain a saccharification mixture under conditions suitable for saccharification. The saccharification unit 1 can be configured to provide for the conversion of cellulosic material with the addition of enzymes. In one embodiment, the saccharification unit 1 is configured for mixing cellulosic material with a liquid and adding a saccharifying enzyme composition to the liquid. In another embodiment, the saccharification unit 1 is configured for saccharification at a variety of pHs and temperatures, but preferably at a pH of 4.5 to 5.0 and at a temperature of about 30 to about 50° C., preferably about 45° C.

The fermentation unit 2 can be any of a variety of apparatus suitable for containing or conducting fermentation. The fermentation unit 1 can be, for example, a vessel in which sugar from cellulosic material can be fermented to ethanol. The fermentation unit 2 can be configured to maintain a fermentation mixture under conditions suitable for fermentation. In one embodiment, the fermentation unit 2 can be configured for fermenting through use of a microorganism, such as yeast. In another embodiment, the fermentation unit 2 can be configured to ferment a saccharification mixture. In yet another embodiment, the apparatus can employ any variety of yeasts that yields a commercially significant quantity of ethanol in a suitable time. Yeast can be added to the apparatus by any of a variety of methods known for adding yeast to a system that conducts fermentation. The fermentation unit 2 can be configured for fermentation for about 24 to 96 hours at a temperature of about 20 to about 40° C.

The saccharification unit 1 and the fermentation unit 2 can be a single, integrated apparatus. In one embodiment, this apparatus is configured to provide higher temperatures early on during simultaneous conversion of cellulosic material to sugars and fermentation of those sugars. In an embodiment, this apparatus is configured to provide lower temperatures later during the simultaneous saccharification and fermentation. The apparatus also may utilize the reagents and conditions described above for saccharification and fermentation, including enzymes and yeast.

The distillation unit 3 can be any of a variety of apparatus suitable for distilling products of fermentation. The distillation unit 3 can be, for example, configured to recover ethanol from the fermentation mixture (“beer”). In one embodiment, the fermentation mixture is treated with heat prior to entering the distillation unit 3. In another embodiment, fractions of large pieces of germ and fiber are removed with a surface skimmer or screen prior to or after entering the distillation unit 3.

The dryer unit 4 can be any of a variety of apparatus suitable for drying solids remaining after distillation (and optional centrifugation, for example, in a centrifuge system). In an embodiment, the dryer unit 4 is configured to dry recovered solids, which can result in production of distiller's dried grain. After the distillation system separates the ethanol from the beer, recovered solids remain. These recovered solids can then be dried in the dryer unit 4. This produces distiller's dried grain and/or distiller's dried grain plus solubles. In one embodiment, the dryer unit 4 can be or include a ring dryer. In another embodiment, the dryer unit 4 can be or include a flash dryer. In yet another embodiment, the dryer unit 4 can be or include a fluid bed dryer.

The distillation step is identical to conventional ethanol process with one exception. The beer from fermentation process is usually lower in ethanol than from a starch process. The lower concentration is a result of the lower solids concentration in the original mash. The concentration of straw mash typically is 10% w/w; it is generally difficult to keep straw suspended in a slurry at concentrations above 10%. Because of the more dilute beer, distillation costs are higher. However the higher distillation costs are offset by lower feedstock costs.

The examples provided herein are for illustration purposes only and are in no means to limit the scope the present invention. Further, all references cited herein are incorporated by reference for all the relevant contents therein.

### EXAMPLES

### Example 1

### Selection of Trichoderma Strain

A number of strains have been tested, including: T. reesei ATCC 56765 (RUT C30), 13631, 24449, 26920, 26921, NRRL 11236, 11480, and 11485; Aspergillus niger ATCC 52172; A. versicolor ATCC 52173 and A. terrus ATCC 52430. All strains tested produced some level of measurable cellulase activity. Of them, ATCC 56765 was selected as the most consistent and highest concentration producer of multiple cellulase and hemicellulase activities and the strain which consistently gave the highest yield in standardized hydrolysis and hydrolysis fermentation of alkali pretreated barley straw.

Selected strains of T reesei were grown in solid substrate culture for further comparison of cellulase production. T. reesei strains were obtained from the ATCC and cultured on PDA agar. Strains were grown in solid substrate culture according to the methods described in example 2. Overall cellulase activity was assayed according to the standardized straw hydrolysis assay also described in Example 2. The ersults are shown below.

| Strain | Glucose concentration mg/ml at 24 hours |
|---|---|
| ATCC 58351 | 9.0 |
| ATCC 58352 | 8.9 |
| ATCC 58353 | 7.1 |
| ATCC 60787 | 14.3 |
| ATCC 56765 | 22.3 |

ATCC 56765 is the strain selected for optimal SSC cellulase production. Other strains do produce cellulase when grown in the SSC process as described. This experiment tested strains grown in the same experiment under equivalent conditions. Seven other strains of T. reesei as well as strains of Aspergillus niger, A. versicolor, A. phoenicis and A. terreus have been tested in separate experiments grown on substrates with varying ratios of components. Although not directly comparable, results of standardized straw assay and selective substrate enzyme assays do demonstrate production of cellulase by multiple strains when grown in the SSC process. In the standardized straw hydrolysis assay 24 hour glucose concentration ranged from 4 to 18 mg/ml with filter paper assay of 20 to 100 units per gram.

### Example 2

### Production of Enzyme Composition Using Solid State Culture

Trichoderma reesei, ATCC 56765 was maintained on PDA agar slants at 4 degrees C. Inoculum cultures were prepared by transferring cells and spores from PDA agar slant to broth containing in grams per liter: NH₄SO₄ 2.8; Kh₂PO₄ 4.0; MgSO₄ 0.6; Urea 0.6; CaCl 0.3; Yeast extract 1.0; soy peptone 1.0; Glucose 10.0; trace elements MnSO₄, FeSO₄, CuSO₄ all less than 0.001. Inoculum culture was grown at 30° C. for 48 hours and used to inoculate solid culture media. Solid culture media contained in grams per kg dry weight: Corn cob beeswing pith and chaff (BPC) 450, barley flour 350, straw (2 mm) 250, wheat germ 250. Dry ingredients were blended and wetted with two liters of salts solution above less glucose per kg of dry substrate, mixed and autoclave at 120° C. 15 psi for 30 minutes then cooled. Substrate was inoculated at 100 ml inoculum culture per kg dry weight substrate equivalent. Inoculated substrate was incubated in a column reactor, 6 inches in diameter by 24 inches long with caps at both ends equipped for inlet and outlet air flow. Columns were aerated at 50 cc/minute air at 90% RH and held at a constant temperature of 30° C. Culture was incubated for 10 days then removed from the column, dried under a flow of dry air at about 25° C., ground in a mill and assayed for enzyme activity by the following methods:

Filter paper assay: 0.1 gram enzyme was added per one filter paper disc. The assay was carried out for two hour at 50° C. The assay was stopped with the addition of DNS reagent, and boiled for 10 min. The results were read at OD 550 nm, One unit equals 1 mg as total reducing sugar.

CMC (carboxymethyl cellulose) assay: 1.0 ml 1% CMC was added to 1.0 ml enzyme water extract (1:1000 dilution) and incubated for 30 minute at 50° C. The reaction was stopped with DNS reagent. One unit equals 1 mg as total reducing sugar.

Cellulose Azure assay: 2 ml of 1% cellulose azure (Calbiochem) was added to 1.0 ml enzyme water extract (1:1000 dilution) and incubated for one hour at 50° C. The reaction was stopped with 1.0 ml 40% acetic acid, and centrifuged. The supernatant is used for OD reading at 595 nm. The units is expressed as the change of OD595 in one hour.

Xylanase assay: 1.0 ml of 0.5% Larchwood xylan was added to 0.5 ml of enzyme water extract (1:1000 dilution) and 0.5 ml buffer, and incubated for 10 minutes at 50° C. The reaction was stopped with addition of DNS reagent. One unit equals 1.0 mg as total reducing sugar, xylose standard.

Standardized straw assay; 1.0 grams alkali pretreated and washed grain straw in 20 ml buffer was autoclaved, and cooled. 0.1 gram enzyme was added and was incubated at 50° C. for 24 hours, and was assayed as glucose or as total reducing sugar. 25 mg/ml glucose represents 50% hydrolysis of pretreated straw.

Dilute alkali pretreatment: 10% straw in 1% NaOH was autoclaved for 15 minutes, and centrifuged. The solids was washed once in 10 volumes of water. The straw solids was recovered and air dried.

All assays was conducted in 0.2M Na Acetate buffer, pH 4.8. DNS assay was conducted according to the method of Miller G L. Anal. Chem. 31:426-429 (1959). As modified, 0.5 ml of sugar solution or hydrolysate diluted as appropriate is incubated with 1.5 ml dinitro salacyclic acid reagent in a boiling water bath for 10 minutes, cooled and read on a spectrophotometer at 550 nm. Total reducing sugar is determined by comparison to a glucose standard.

Results are shown in Table 1. Data compares solid culture enzyme preparation labeled SSC with a commercial cellulase Novozymes Celluclast 1.5 L assayed on an equal weight basis by the same methods.

TABLE 1
Selective Substrate Assays of SSC Enzyme Preparations
Assay Substrate

| | Filter | | Cellulose | | Straw |
|---|---|---|---|---|---|
| | Paper | CMC | Azure | Xylan | Gulucose |
| | units/gr | units/gr | ΔOD₅₉₅ | units/gr | mg/ml |
| SSC | 152 | 118 | 0.263 | 102 | 25 |
| Novo | 144 | 109 | 0.223 | — | 22.4 |

MMP enzyme bench scale production test 500 gram SSC Novo Celluclast 1.5 L cellulase from Trichoderma reesei

### Example 3

### Cellobiase Activity in Solid Culture Enzyme Preparations

One advantage of the composition of the enzyme produced using solid culture technology of the present invention is production of cellobiase activity in the same culture as the endoglucanase and cellobiohydrolase activities. The cellobiase converts cellobiose to glucose which can be fermented by common yeast strains. Enzyme preparations produced according to the method described in example 2 above and assayed for cellobiase activity.

Assay procedure: A solution of cellobiose 2 mg/ml was treated with 10% w/w solid culture derived enzyme and incubated at 30° C. for 30 minutes. The enzyme treatment was compared to the same solution of cellobiose incubated without addition of enzyme. After incubation tubes were assayed for total reducing sugar by the DNS method. This method assays only the reducing end of the molecule measures the two glucose residue cellobiose molecule as a single reducing sugar. If cellobiose is hydrolyzed to monomeric glucose molecules the reducing sugar will increase as both glucoses now react with the DNS reagent. Incubation of the cellobiose solution with solid culture enzyme increased total reducing sugar concentration from 2 mg/ml to 3.5 mg/ml indicating nearly complete conversion of cellobiose to glucose.

### Example 4

### Pilot Scale Solid Substrate Culture for Cellulase/Hemicellulase Enzyme Preparation

The four component substrate described in example 1 was prepared in lots of approximately 20 kg, wetted with the nutrient solution described in example 2. The moist substrate was placed in autoclave bags and autoclaved for approximately one hour at 125° C., cooled and inoculated at 10% v/w of a 48 hour culture of T. reesei ATCC 56765 grown in the broth medium described in example 1. The inoculated substrate was placed on screen trays in a commercial solid culture incubation chamber and incubated for 10 days at about 30° C. under a constant flow of air at 90% RH. After 10 days the cultures were dried and ground and used as the cellulase enzyme preparation as describe in Example 5 below.

### Example 5

### Test Runs Using the SSC Cellulase to Make Ethanol From Grass Straw

The straw was ground in an air swept pulverizer to pass a 60 mesh screen. Pulverized straw was treated with 1% NaOH at 80° C. and 9 psi. The wet straw was passed through a screw press to remove the “black liquor”. (Black liquor is a caustic solution containing solublized lignin and other materials.) The wet pulp was washed and passed through a second and then third screw press to remove additional solubles and to help adjust pH. The squeezed pulp was fed to a 200 gallon stirred fermentation tank. Water, enzyme, yeast and acid were added to the fermentation tank. The fermentation tank was maintained at approximately 68° F., or room temperature. Enzyme used in these experiments was prepared as described in example 4 and used at a ratio of about 10% w/w enzyme to straw on a dry weight basis.

The pretreated straw was used as a stand-alone feedstock in three separate simultaneous hydrolysis and fermentation process to produce a beer containing 3.46% ethanol or 66 gallons ethanol per dry weight ton of delignified straw. The solids were separated from the liquid using a screw press. The separated water contained 3.46% ethanol. Data showed a 95% conversion of the cellulose to ethanol. The yeast employed in these experiments was standard distillery yeast (Saccharomyces cerevisiae) which does not ferment pentose sugars from hemicellulose. The assumption is that the ethanol production is a result of cellulose conversion although there may have been a small contribution from galactose from the hemicellulose fraction. Ethanol concentrations were measured at 48, 56, 72, and 78 hours. Maximum ethanol concentration occurred at 72 hours. Incorporating a simultaneous hydrolysis and fermentation step eliminates the potential for sugar feedback inhibition during hydrolysis.

TABLE 2

| | weight of | | |
|---|---|---|---|
| | straw | enzyme | EtOH yield |
| | in slurry | loading | at 72 hours |
| Blended straw run 1 | 166 lbs | 17 lbs | 65.7 gal/ton |
| Blended straw run 2 | 166 lbs | 17 lbs | 65.5 gal/ton |
| Blended straw run 3 | 199 lbs | 20 lbs | 65.9 gal/ton |

### Example 6

### Performance of Solid Culture Cellulase Preparations Under Commercial Hydrolysis Process Conditions

Solid culture cellulase preparations were prepared according to the method described in example 2 for culture of T. reesei on the blended substrate in solid substrate culture. Experiments comparing hydrolysis of washed and unwashed alkali pretreated straw demonstrated that the solid culture enzyme preparation performs in the presence of lignin or any inhibitory compounds generated during pretreatment. Table 3 shows results of hydrolysis of unwashed, and washed alkali pretreated straw. Unwashed straw contains all of the potentially inhibitory compounds formed during alkali pretreatment.

TABLE 3
Performance of SSC Enzyme Under Process Relevant Conditions
Hydrolysis of Unwashed and Washed Alkali Pretreated Straw

Reducing Sugars, mg/ml

| | | |
|---|---|---|
| Pretreated | 4 Hour | 24 Hour |

Unwashed pH Adjusted

| | | |
|---|---|---|
| Mild | 11.4 | 18.1 |
| Severe | 11.3 | 15.6 |

Washed pH Adjusted Solids

| | | |
|---|---|---|
| Mild | 10.6 | 17 |
| Severe | 9.9 | 16.9 |

The experiment used 7.5% straw solids in one of two pretreatment conditions: mild was 1% NaOH, 80° C. for 1 hour; and severe was 4% NaOH for 1 hour, 15 psi, at 121° C. In the unwashed treatment there was no separation of solids, the pretreated straw suspension was adjusted to pH 4.8 with addition of sulfuric acid. Enzyme was added directly to the suspension. In the washed solids, the pretreated straw suspension was centrifuged, re-suspended in water, pH was adjusted to 4.8 and the suspension centrifuged a second time. Solids were then re-suspended in water pH 4.8 to approximately the same solids content as the unwashed sample. The re-suspended washed solids were then treated with SSC enzyme. Analysis of supernatant from the pretreatment and pretreatment wash showed minimal total reducing sugars confirming that the alkali pretreatment results in minimal hemicellulose hydrolysis.

There was no apparent inhibition of enzyme activity in the unwashed pretreated, pH adjusted sample. Total reducing sugar concentrations in the unwashed and washed samples were approximately equal. (Volumes and solids concentrations were adjusted to be close to the 7.5% starting concentration but could not be exactly comparable

What is claimed is:

1. A method of producing ethanol, comprising:
(1) providing an SSC Cellulase enzyme composition made by a solid state culture process, said process comprising:
(i) steaming a substrate comprising cob, straw, barley, and wheat germ;
(ii) growing a fungus on said substrate for a first period of time in a growth chamber; and
(iii) harvesting the enzyme composition;
(2) providing a mash that is adjusted to pH 4 to 6;
(3) mixing said mash with said SSC Cellulase enzyme composition and yeast; and
(4) incubating for a period of fermentation time under a temperature between 20 to 40° C. to produce ethanol.

2. The method according to claim 1, wherein said fungus is the strain of Trichoderma reesei, ATCC-57560.

3. The method according to claim 1, wherein said method further comprising collecting said ethanol.

4. The method according to claim 3, further comprises distilling said ethanol.

5. The method according to claim 1, wherein said mash has up to 40% solid.

6. The method according to claim 1, wherein said mash is adjusted to pH about 4.8.

7. The method according to claim 1, wherein said temperature is about 35° C.

8. The method according to claim 1, wherein said mash comprises cellulose material with lignin, and wherein said cellulose is pretreated using alkaline processes to remove said lignin.

9. The method according to claim 1, wherein said incubating step comprises simultaneously hydrolysis and fermentation.

10. The method according to claim 1, wherein said fungus is selected from a group consisting of T. reesei, Aspergillus niger, A. phoenicis, A oryzae, A awamori, Rhizopus oryzae, R. microsporus, Acidothermus cellulyticus and Trichoderma koningii, Trichoderma viride. T. harzianum, Fusarium oxysporum Penicillum pupurogenum, and Myceliophthora sp., Lentinous.

11. A method of making enzyme composition comprising.
(1) providing a solid fermentation substrate; and
(2) growing a Trichoderma reesei ATCC-57560 on said substrate for a first period of time in a growth chamber to produce an enzyme composition.

12. The method according to claim 11, further comprising harvesting said enzyme composition.

13. The method according to claim 11, wherein said substrate comprises barley.

14. The method according to claim 11, wherein said substrate has undergone heat treatment.

15. The method according to claim 14, wherein said heat treatment is steaming.

16. The method according to claim 15, wherein said steaming is conducted at ambient pressure.

17. A composition comprising:
(1) a Trichoderma reesei ATCC-57560; and
(2) a substrate comprises corn cob, straw, barley, and wheat germ; and
(3) endo and exo acting cellulase, cellobiase, and xylanses.

18. The composition according to claim 17, wherein said Trichoderma reesei produces endo and exo acting cellulase, cellobiase, and xylanses at a pH optima of 4.8.

19. A composition comprising:
(1) an SSC Cellulase enzyme composition;
(2) a yeast; and
(3) mash.

20. The composition according to claim 19, wherein said SSC Cellulase enzyme composition is made by a solid state culture process, said process comprising:
(i) steaming a substrate to adjust moisture and reduce contamination from indigenous microorganisms, wherein said substrate comprises cob, straw, barley, and wheat germ;
(ii) growing a Trichoderma reesei, ATCC-57560 on said substrate for a first period of time in a growth chamber; and
(iii) harvesting the enzyme mush comprises the mixture of said Trichoderma reesei and substrate.
